(19) United States
(12) Patent Application Publication
(10) Pub. No.: US 2017/0290954 A1
(43) Pub. Date: Oct. 12, 2017

(54) COMPOSITIONS AND METHODS FOR TREATING SPINAL CORD INJURY

(71) Applicant: B. G. Negev Technologies and Applications Ltd., at Ben-Gurion University, Beer Sheva (IL)

(72) Inventors: Smadar COHEN, Beer Sheva (IL); Dasa CIZKOVA, Kosice (SK)

(21) Appl. No.: 15/480,491

(22) Filed: Apr. 6, 2017

Related U.S. Application Data
(60) Provisional application No. 62/319,339, filed on Apr. 7, 2016.
(60) Provisional application No. 62/384,213, filed on Sep. 7, 2016.

(51) Int. Cl. A61L 27/54; A61L 27/48; A61L 27/52; A61L 27/22

(52) U.S. Cl. CPC A61L 27/54; A61L 27/227; A61L 27/48; A61L 27/52; A61L 2430/32; A61L 2300/414

(57) ABSTRACT

The present invention provides a method for treating spinal cord injury and repairing or regenerating a damaged spinal cord tissue comprising administering a composition comprising a sulfated alginate or sulfated hyaluronan optionally affinity-bound to one or more growth factors.

### FIELD OF THE INVENTION

The present invention provides a method for treating spinal cord injury and repairing or regenerating a damaged spinal cord tissue comprising administering a composition comprising sulfated alginate or sulfated hyaluronan optionally affinity-bound to one or more growth factors.

### BACKGROUND OF THE INVENTION

Spinal cord injury (SCI) involves a multifactorial process that initiates pathological cellular and molecular responses resulting in limited spontaneous axonal regeneration. Clinical symptoms following trauma can vary in severity, but usually lead to complete paralysis and spasticity. The development of a safe and efficient treatment for spinal cord injuries is greatly complicated by the existence of a highly complex injury environment.

Over the past decades various strategies have been proposed including inflammatory processes and suppression of edema, promotion of axonal regeneration through the decrease of inhibitory molecules, transplantation of stem cells to replace lost tissue, or enhancement of endogenous repair with trophic factor support and rehabilitative training. All these strategies were developed to target specific pathological players during secondary damage, whereas nowadays a combinatorial approach integrating biomaterial scaffolds, cell transplantation and molecule delivery seems to be more promising for regeneration and functional recovery.

The alginate scaffold is a suitable biomaterial construct providing a cellular mechanical framework of polysaccharide chains that gels by ionic cross linking after mixing aqueous alginate solution with divalent cations such as Ca2+. Natural substrate isolated from the wall of brown seaweed represents a non-toxic/non-inflammatory, highly porous scaffold with relatively low cost. Alginate hydrogel has been widely used for drug or cell delivery as an injectable vehicle capable of filling cavities in the injured spinal cord, and of providing the substrate for axon attachment and re-growth.

Studies have shown that the adult spinal cord harbours a population of multipotent neural precursor cells (NPCs), which is further increased by injury, but they are insufficient to replace lost neuroglia populations.

Accordingly, there exists a need for a therapeutically effective method for treating SCI which enhances the survival, and integration of endogenous NPCs into functional neural circuitry in situ.

### SUMMARY OF THE INVENTION

In one aspect, the invention provides a method for repairing or regenerating a damaged spinal cord tissue in a subject, the method comprising: administering to said subject a composition comprising: a polysaccharide, and a bioactive polypeptide or a protein molecule operably linked to said polysaccharide, wherein said polysaccharide is an sulfated alginate or sulfated hyaluronan and said molecule is growth factor, thereby repairing or regenerating said damaged spinal cord tissue in said subject.

In another aspect, the invention provides a method for treating a spinal cord injury in a subject, the method comprising: administering to said subject a composition comprising: a polysaccharide, and a bioactive polypeptide or a protein molecule operably linked to said polysaccharide, wherein said polysaccharide is sulfated alginate or sulfated hyaluronan and said molecule is growth factor, thereby treating said spinal cord injury in said subject.

In yet another aspect, the invention provides a method for promoting the regrowth of one or more axons in a subject with a spinal cord injury, the method comprising the step of: administering to said subject a composition comprising a polysaccharide and a polypeptide operably linked to said polysaccharide, wherein said polysaccharide is a sulfated alginate or a sulfated hyaluronan, and wherein said polypeptide is a growth factor, thereby promoting the regrowth of said axons in said subject.

In yet another aspect, the invention provides a method for increasing the number of surviving neurons, sensory fibres, or a combination thereof in a subject with a spinal injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is a growth factor, thereby increasing the number of surviving neurons, sensory fibres, or a combination thereof in said subject.

In yet another aspect, the invention provides a method for increasing the number of blood vessels in a central lesion in a subject with a spinal injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is a growth factor, thereby increasing the number of blood vessels in said subject with a spinal injury.

In yet another aspect, the invention provides a method for repairing or regenerating damaged spinal cord tissue in a subject, the method comprising the step of: administering to said subject a composition comprising an alginate, alginate sulfate, or hyaluronan sulfate, or a combination thereof, and lacking a bioactive polypeptide, thereby repairing or regenerating said damaged spinal cord tissue in said subject

In yet another aspect, the invention provides a method for treating a spinal cord injury in a subject, the method comprising the step of: administering to said subject a composition comprising an alginate, alginate sulfate, or hyaluronan sulfate, or a combination thereof, and lacking a bioactive polypeptide, thereby treating said spinal cord injury in said subject.

In yet another aspect, the invention provides a method for promoting the regrowth of one or more axons in a subject with a spinal cord injury, the method comprising the step of: administering to said subject a composition comprising an alginate, alginate sulfate, or hyaluronan sulfate, or a combination thereof, and lacking a bioactive polypeptide, thereby promoting the regrowth of said axons in said subject.

In yet another aspect, the invention provides a method for increasing the number of surviving neurons, sensory fibres, or a combination thereof in a subject with a spinal injury, the method comprising the step of: administering to said subject a composition comprising an alginate, alginate sulfate, or hyaluronan sulfate, or a combination thereof, and lacking a bioactive polypeptide, thereby increasing the number of surviving neurons, sensory fibres, or a combination thereof in said subject.

In yet another aspect, the invention provides a method for increasing the number of blood vessels in a central lesion in a subject with a spinal injury, the method comprising the step of: administering to said subject a composition comprising an alginate, alginate sulfate, or hyaluronan sulfate, or a combination thereof, and lacking a bioactive polypeptide, thereby increasing the number of blood vessels in said subject with a spinal injury.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1A. Illustration of the bregma and lambda anatomical location.

FIG. 1B. Illustration of the bregma and lambda anatomical location with four openings for BDA administration targeting the sensorimotor cortex.

FIG. 2. Schematic drawing of individual experimental steps of present study.

FIG. 3. Concept of dissected spinal cord segments consisting of rostral (Th5-7), central (Th8-9), caudal (Th10-12) segments processed for immunohistochemical analyses.

FIG. 4A. Schematic illustration of areas for cells quantification. A Quantification of NeuN was performed bilaterally in 3 squares (200×200 μm) in the Laminae I-IV (dorsal horn) (1), Laminae IV-V (intermediate zone) (2) and Laminae VIII-IX (ventral horn) (3). Scale bars: 500 μm, 10 μm.

FIG. 4B. Evaluation of Iba1 positivity was performed bilaterally within 3 squares (150×150 μm) in grey matter—Lamina VII (1) and white matter—lateral (2) as well as ventral funiculi (3). Scale bars: 500 μm, 10 μm.

FIG. 5A. Functional recovery of hindlimb motor function following SCI in Sham, SCI+SAL, SCI+ALG and SCI+ALG+GFs experimental groups. *P<0.05 indicates significant differences among the experimental groups.

FIG. 5B. Functional recovery of sensory functions following SCI in Sham, SCI+SAL, SCI+ALG and SCI+ALG+GFs experimental groups.

FIG. 6A. Morphometric analyses of cavity length in experimental groups showed significant reduction after intraspinal injection of biomaterial alginate (**P<0.01, ***P<0.001). Cavity size was expressed by mm in injured experimental groups (SCI+SAL, SCI+ALG and SCI+ALG+GFs) relative to Sham spinal cord without cavitations represent by zero.

FIG. 6B. Morphometric analyses of cavity size in experimental groups showed significant reduction after intraspinal injection of biomaterial alginate (**P<0.01, ***P<0.001). Cavity size was expressed by mm in injured experimental groups (SCI+SAL, SCI+ALG and SCI+ALG+GFs) relative to Sham spinal cord without cavitations represent by zero.

FIGS. 7A-7C. Stereological analyses of NeuN positive cells in Laminae I-IV (FIG. 7A), Laminae IV-V (FIG. 7B) and Laminae VIII-IX (FIG. 7C) rostral (left graph) and caudal (right graph) from the lesion site after SCI and treatment. Number of NeuN labeled neurons in all studied areas increased after pure and enriched alginate administration compared to saline. Among the experimental groups we observed statistical differences (***P<0.001, **P<0.01, *P<0.05).

FIG. 8. Quantification of ChAT labeled motoneurons in the Laminae VIII-IX. Marked depletion of motoneurons was observed caudally to the lesion site when compared with segment located rostrally. Significantly higher number of ChAT positive neurons was detected after delivery of enriched alginate in both studied segments (rostral, top; caudal, bottom) (***P<0.001, *P<0.05).

FIG. 9. Representative transverse sections revealing ChAT positive motoneurons located rostrally and caudally to the lesion site following SCI in Sham, SCI+SAL, SCI +ALG and SCI+ALG+GFs experimental groups. Significant decrease of ChAT immunohistochemical staining was observed in ventral horns caudally to the lesion site (lower panel) following SCI and saline delivery. Injected alginate supports survival of ChAT positive motoneurons. Scale bar=500 μm.

FIGS. 10A-10C. Distribution of synaptophysin positive vesicles (red) in the area of ChAT positive motoneurons (green) of the ventral horns. The density of vesicles after ALG+GFs treatment (FIG. 10C) is increased compared to both sham (FIG. 10A) and ALG without GFs group (FIG. 10B).

FIG. 10D. Quantification has revealed statistical significance only between ALG+GFs group and other experimental groups (SHAM, SCI+SAL, SCI+ALG). (**P<0.01, ***P<0.001). Scale bar=50 μm.

FIGS. 11A-11B. CGRP expression in transverse rostral (FIG. 11A) and caudal (FIG. 11B) sections of dorsal horn/thoracic spinal cords from Sham, SCI+SAL, SCI+ALG and SCI+ALG+GFs groups 49D post-injury. Representative photographs demonstrate differences in CGRP expression, particularly in number and length of CGRP positive fibers among the experimental groups. Note, enhanced growth and branching of CGRP fibers from dorsal horn to Laminae III-V and VII in SCI+ALG and SCI+ALG+GFs groups (A3, B3, A4, B4) when compared to sham and saline rats (A1, B1, A2, B2). Scale bar=500 μm. Lower panel shows higher magnification from corresponding regions. Scale bar=250 μm.

FIG. 12A. Number of CGRP positive fibers. Quantitative analyses of CGRP positive fibers number showed no significant differences between individual experimental groups.

FIG. 12B. Length of CGRP positive fibers. Quantitative analyses of CGRP positive fibers number demonstrated statistically significant differences between individual experimental groups in the length of CGRP positive fibers between Sham and other experimental groups (SCI+SAL, SCI+ALG, SCI+ALG+GFs) (*P<0.05, **P<0.01, ***P<0.001).

FIG. 13. BDA labeling in the representative sagittal sections in Sham, SCI+SAL, SCI+ALG and SCI+ALG+GFs groups 49D post injury. Representative pictures and corresponding details (boxed areas) illustrate growth potential of CST fibers after SCI and individual treatments. Increased positivity of BDA was seen after alginate administration (SCI+ALG and SCI+ALG+GFs groups) where CST axons were growing around the lesion site towards disconnected caudal segment. Scale bar=500 μm.

FIG. 14. Quantification of BDA positivity in sagittal section of spinal cord. Administration of alginate enriched with growth factors leads to the significant (*P<0.05) increase of BDA labeled fibers when compared to saline treatment.

FIG. 15. Representative transverse sections of Iba1 expression from caudal and rostral segments of spinal cord. Sections illustrate changes in activation and cell morphology after saline and alginate treatment. Baseline expression of non-active microglia in gray and white matter was observed in Sham animals. Strong activation of microglia characterized by enlarged round-shaped perikaryon with truncated, dick and ramified processes was detected in SCI+SAL group. Alginate treatment (SCI+ALG and SCI+ALG+GFs groups) inhibited activation of Iba1 positive cells. Scale bars=500 μm, 10 μm (higher magnification of boxed area).

FIG. 16. Densitometric analyses of Iba1 positivity through the 1.6 cm sagittal sections of spinal cord Sham tissue exhibits limited levels of Iba1 expression and low background staining. The highest expression of Iba1 positivity was detected following injury and saline treatment. Intraspinal administration of pure and enriched alginate showed significant differences in Iba1 expression between experimental groups mainly caudally from the lesion site (*** P<0.001, ** P<0.01, * P<0.05).

FIGS. 17A-17B. Baseline expression of GFAP-positive astrocytes with the characteristic round nuclei and slender, long processes distributed throughout both white and gray matter was revealed in Sham animals 49D post-injury. SCI and vehicle/saline treatment induced increase in GFAP immunohistochemical staining associated with cellular transformation; swollen hypertrophic appearance and short, thick processes indicating activated phenotype. Similarly alginate slightly increased the GFAP density but positive cells were without hypertrophic appearance. Scale bars=100 μm (FIG. 17A), 50 μm (FIG. 17B).

FIG. 18A. Densitometric analyses of GFAP positivity in Sham, SCI+SAL, SCI+ALG and SCI+ALG+GFs groups 49D post-injury. A significant difference among experimental groups and individual parts of spinal cord was detected in 1.6 cm sagittal sections from central lesion (***P<0.001, **P<0.01, *P<0.05).

FIG. 18B. Densitometric analyses of GFAP positivity in Sham, SCI+SAL, SCI+ALG and SCI+ALG+GFs groups 49D post-injury. GFAP expression in transverse sections from rostral and caudal segments (0.8 cm from the lesion site) was without visible significance between experimental groups.

FIG. 19. Effect of SCI and treatment on spontaneous recovery of vWF positive blood vessels. The longitudinal sections of injured spinal cord showed formation of new blood vessels in close vicinity of lesion and also in lesion site after alginate treatment. vWF expression was enhanced not only in the penetrating blood vessel but also in cell bodies found close to the injury site, but mainly in rostral direction. The sham tissue exhibits homogeneous distribution of vWF staining throughout the whole section with visible structure of vessels in white matter. Scale bar: 500 μm.

### DETAILED DESCRIPTION OF THE INVENTION

The invention relates to a polysaccharide-based controlled release composition and uses thereof. Specifically, the invention relates to a composition comprising a sulfated alginate or a sulfated hyaluronan in combination with one or more growth factors for use in repairing or regenerating a damaged tissue of spinal cord.

In one aspect, provided herein is a composition comprising: a polysaccharide and a polypeptide operably linked to said polysaccharide, wherein said polysaccharide is an alginate, sulfated alginate, or sulfated hyaluronan, and wherein said polypeptide is a growth factor. In one embodiment, the polypeptide is a bioactive polypeptide.

Bioactive Polypeptides

In one embodiment, the term “bioactive polypeptide” as used herein refers to a polypeptide exhibiting a variety of pharmacological activities in vivo and include, without being limited to, growth factors, cytokines, chemokines, angiogenic factors, immunomodulators, hormones, and the like. In the present application, the terms “polypeptide” and “proteins” are used interchangeably. In one embodiment, the bioactive polypeptide may be a positively-charged polypeptide.

In one embodiment, the bioactive polypeptide is a growth factor. The present invention encompasses all the known isoforms of the growth factor, as well as their fragments, mutants, homologs, analogs and allelic variants. Each possibility represents a separate embodiment of the present invention. In one embodiment, the growth factor is a mammalian growth factor.

Any suitable one or more growth factors can be used. In one embodiment, the growth factor is an Epidermal Growth Factor (EGF). In another embodiment, the growth factor is a Fibroblast Growth Factor (FGF). In one embodiment, FGF is basic FGF (bFGF). In another embodiment, the growth factor is EGF and an FGF such as bFGF. In another embodiment, the growth factor is insulin-like growth factor-1 (IGF-1).

In some embodiments, a polypeptide or growth factor of the present invention is a pro-angiogenic growth factor. In one embodiment, the pro-angiogenic growth factor is hepatocyte growth factor (HGF), vascular endothelial growth factor (VEGF), platelet-derived growth factor (PDGF), fibroblast growth factor (FGF), or a combination thereof. In one embodiment, FGF is acidic FGF (aFGF).

In some embodiments, a polypeptide or growth factor of the present invention is neuroprotective. In one embodiment, the neuroprotective polypeptide is brain-derived neurotrophic factor (BDNF), glial cell-derived neurotrophic factor (GDNF), nerve growth factor (NGF), or a combination thereof. In one embodiment, the NGF is neurotrophin-3 (NT-3).

In some embodiments, the composition of the invention may comprise one or more additional bioactive polypeptides. In one embodiment, the additional polypeptide is a growth factor or morphogen. Examples of additional bioactive polypeptides include, but are not limited to, insulin-like growth factor-1 (IGF-1), transforming growth factor β1 (TGF-β1), bone Morphogenetic Protein 4 (BMP4), insulin, glatiramer acetate (also known as Copolymer 1 or Cop 1), antithrombin III, interferon (IFN)-γ (also known as heparin-binding protein), somatostatin, erythropoietin, luteinizing hormone-releasing hormone (LH-RH) and interleukins such as IL-2 and IL-6.

In one embodiment, the additional bioactive polypeptide is a heparin-binding protein or polypeptide. The term “heparin-binding protein or polypeptide” may refer to a protein having clusters of positively-charged basic amino acids and form ion pairs with specially defined negatively-charged sulfo or carboxyl groups on the heparin chain (See Capila and Linhardt, 2002).

In one embodiment, the additional polypeptide is transforming growth factor β1 (TGF-β1), TGF-β3, antithrombin III (AT III), thrombopoietin (TPO), serine protease inhibitor (SLP1), C1 esterase inhibitor (C1 INH), Vaccinia virus complement control protein (VCP), a fibroblast growth factor (FGF), bFGF, aFGF, a FGF receptor, vascular endothelial growth factor (VEGF), hepatocyte growth factor (HGF), insulin-like growth factor (IGF), IGF-1, a platelet-derived growth factor (PDGF), PDGF-BB, PDGF-AA, epidermal growth factor (EGF), CXC chemokine ligand 4 (CXCL4), stromal cell-derived factor-1 (SDF-1), interleukin-6 (IL-6), IL-8, IL-10, Regulated on Activation, Normal T Expressed and Secreted (RANTES), monocyte chemoattractant protein-1 (MCP-1), macrophage inflammatory peptide-1 (MIP-1), lymphotactin, fractalkine, an annexin, apolipoprotein E (ApoE), immunodeficiency virus type-1 (HIV-1) coat protein gp120, cyclophilin A (CypA), Tat protein, viral coat glycoprotein gC, gB or gD of herpes simplex virus (HSV), an envelope protein of Dengue virus, circumsporozoite (CS) protein of Plasmodium falciparum, bacterial surface adhesion protein OpaA, 1-selectin, P-selectin, heparin-binding growth-associated molecule (HB-GAM), thrombospondin type I repeat (TSR), peptide myelin oligodendrocyte glycoprotein (MOG), amyloid P (AP), or BMP-2. In one embodiment, the additional polypeptide exhibits angiogenic activity. In one embodiment, the polypeptide exhibiting angiogenic activity is VEGF, bFGF, aFGF, PDGF-RR or a combination thereof. In one embodiment, the polypeptide exhibits anti-inflammatory activity. In one embodiment, the polypeptide is interleukin-1 receptor antagonist (IL-1RA).

The present invention provides sustained release of polypeptides that is maintained over a period of about 10 days, 15 days, 30 days, 60 days, 90 days, 180 days, 270 days, or 365 days. In one embodiment, the sustained release period is about 10 days. In another embodiment, the sustained release period is about 15 days. In yet another embodiment, the sustained release period is about 30 days. In a further embodiment, the sustained release period is about 60 days. In another embodiment, the sustained release period is about 90 days. In yet another embodiment, the sustained release period is about 180 days. In a further embodiment, the sustained release period is about 270 days. In yet further embodiment, the sustained release period is about 365 days.

In some embodiments, the invention provides for the localized release of the polypeptide(s). In other embodiments, the invention provides for the systemic release of the polypeptide(s).

In another example, the additional bioactive polypeptide is a positively charged polypeptide. The term “positively charged polypeptide” may refer to a polypeptide or protein that has a positive net charge at physiological pH of about pH=7.5. The positively charged polypeptides are well known in the art. Examples of a positively charged polypeptide include, but not limited to, insulin, glatiramer acetate (also known as Copolymer 1 or Cop 1), antithrombin III, interferon (IFN)-γ (also known as heparin-binding protein), somatostatin, erythropoietin, luteinizing hormone-releasing hormone (LH-RH) and interleukins such as IL-2 and IL-6.

In one embodiment, the polysaccharide and the polypeptide are each present in an amount effective to treat a spinal cord injury in a subject, repair or regenerate a damaged spinal cord tissue in a subject, to promote the regrowth of one or more axons in a subject, increase the number of surviving neurons, sensory fibres, or a combination thereof in a subject with a spinal injury, increase the number of blood vessels in a central lesion in a subject with a spinal injury, etc.

The concentration of one or more bioactive polypeptides in the matrix may be approximately 10⁻⁴, 10⁻³, 0.01, 0.1, or 1% (w/v). In another embodiment, the concentration is approximately 2×10⁻⁴, 3×10⁻⁴, 4×10⁻⁴, 5×10⁻⁴, 6×10⁻⁴, 7×10⁻⁴, 8×10⁻⁴, or 9×10⁻⁴% (w/v). In another embodiment, the concentration is 2×10⁻³, 3×10⁻³, 4×10⁻³, 5−10⁻³, 6−10⁻³, 7×10⁻³, 8×10⁻³, or 9×10⁻³% (w/v). In another embodiment, the concentration is 2×10⁻², 3×10⁻², 4×10⁻², 5×10⁻², 6×10⁻², 7×10⁻², 8×10⁻², or 9×10⁻²% (w/v). In some embodiments, the concentration may range approximately 10⁻⁴-1%, 10⁻⁴-0.1%, 10⁻⁴-0.01%, 10⁻⁴-10⁻³%, or 10⁻³-0.1% (w/v).

Polysaccharides

A polysaccharide of the compositions and methods/uses of the present invention can be any suitable polysaccharide that facilitates repair, regeneration, or replacement of damaged or diseased tissue. Examples of such polysaccharides include, for example, but are not limited to, an alginate, a chitosan, and a glycosaminoglycan. In one embodiment, the present invention provides methods for treating spinal cord damage comprising administering a composition comprising alginate and lacking sulfated alginate or other sulfated polysaccharide, wherein the alginate is operably linked to a polypeptide of interest, which, in one embodiment, is a growth factor.

Alginic acid is a linear polysaccharide obtained from brown algae and seaweed and consist of β-1,4-linked glucuronic and mannuronic acid units. As used herein, the term “alginate” refers to a polyanionic polysaccharide copolymer derived from sea algae (e.g., Laminaria hyperborea, L. digitata, Eclonia maxima, Macrocystis pyrifera, Lessonia nigrescens, Ascophyllum codosum, L. japonica, Durvillaea antarctica, and D. potatorum) and which includes β-D-mannuronic (M) and α-L-guluronic acid (G) residues in varying proportions.

In one embodiment, an alginate suitable for use in the present invention may have a ratio between α-L-guluronic acid and β-D-mannuronic in one embodiment, ranging between 1:1 to 3:1, and in another embodiment, between 1.5:1 and 2.5:1. In a particular example, the ratio between α-L-guluronic acid and β-D-mannuronic is about 2:1.

In one embodiment, an alginate suitable for use in the present invention has a molecular weight ranging, in one embodiment, between 1 to 300 KDa, in another embodiment, between 5 to 200 KDa, in another embodiment, between 10 to 100 KDa, and in another embodiment, between 20 to 50 KDa.

Alginate undergoes gelation in the presence of bivalent cations, such as Ca²⁺ and Ba²⁺. In the pharmaceutical/medicinal fields, it is used successfully as encapsulation material, mostly for cells (bacterial, plant and mammalian cells). For molecules, it is much less effective, and even macromolecules in size of 250 kDa are rapidly released from alginate hydrogel systems. In particular, biological molecules of interest such as cytokines, growth factors, with sizes ranging between 5 to 100 kDa, are rapidly released.

Sulfated Polysaccharides

In a particular embodiment, the polysaccharide is a sulfated polysaccharide (e.g., alginate sulfate). The invention also encompasses other polymers that facilitate repair, regeneration, or replacement of damaged or diseased tissue. These other polymers may include, for example, but are not limited to, collagen, poly(α-hydroxy acids) (e.g., poly(lactic acid), poly(glycolic acid), and poly(lactic-co-glycolic acid)), pseudo-poly(amino acids), polyhydroxybutyrate, polyethylene glycol, fibrin, and gelatin.

In one embodiment, the polysaccharide as described herein is a sulfated polysaccharide, which in one embodiment, is a synthetically sulfated polysaccharide. In accordance with the present invention, the sulfated polysaccharides forming the bioconjugate may be composed of different recurring monosaccharide units, may be of different lengths, and may have different types of bonds linking said units. The sulfated polysaccharides may be linear as, for example, sulfated cellulose, branched as, for example, sulfated glycogen, and may vary in length; for example, it may be as small as a sulfated tetra- or tri-saccharide. The suitable sulfated polysaccharide may be a homopoly saccharide including, but not limited to, starch, glycogen, cellulose, chitosan, or chitin or a heteropolysaccharide including, but not limited to, alginic acid (alginate) salts and hyaluronic acid.

According to the present invention and in one embodiment, the sulfated polysaccharides may comprise uronic acid residues such D-glucuronic, D-galacturonic, D-mannuronic, L-iduronic, and L-guluronic acids. Examples of polysaccharides comprising uronic acid residues include, but are not limited to, alginic acid salts, in one embodiment, sodium alginate, pectin, gums and mucilages from plant sources; and glycosaminoglycans (GAGs) from animal sources including hyaluronic acid (hyaluronan). The sulfated polysaccharides comprising uronic acid can be chemically sulfated or may be naturally sulfated polysaccharides.

In one embodiment, the sulfated polysaccharide is alginate sulfate. In another embodiment, the sulfated polysaccharide is hyaluronan sulfate.

Hyaluronic acid is composed of repeating dimeric units of glucuronic acid and N-acetyl glucosamine and forms the core complex proteoglycans aggregates found in the extracellular matrix.

In one embodiment, the concentration of polysaccharide compared to the total weight of the composition of the present invention may be approximately 1, 5, 10, 15, 20, 25, 30, 35, 40, 45, 50, 55, 60, 65, 70, 75, 80, 85, 90, 95, or 100% (w/w). In some embodiments, the concentration may range approximately 5-95%, 10-90%, 20-80%, 30-70%, or 40-60% (w/w).

In one embodiment, a hyaluronan sulfate of the present invention is highly sulfated. In another embodiment, a hyaluronan sulfate of the present invention comprises 1-4 sulfate groups per dimer. In one embodiment, a hyaluronan sulfate of the present invention comprises 1-6 sulfate groups per dimer. In one embodiment, a hyaluronan sulfate of the present invention comprises 2-3 sulfate groups per dimer.

Thus, in one embodiment, the present invention provides a composition comprising a matrix comprising sulfated hyaluronan sulfated at C-6 and C-4 of N-acetyl-D-glucosamine. In another embodiment, the present invention provides a composition comprising a matrix comprising sulfated hyaluronan sulfated at C-6 and C-4 of N-acetyl-D-glucosamine and at C-2 and C-3 of the glucuronic acid.

The present invention also contemplates a mixture of sulfated and unsulfated polysaccharides, for example alginate and alginate sulfate. According to the present invention the proportion of sulfated polysaccharide may range from about 1% to about 40% of the total polysaccharide by weight, in one embodiment, from about 3% to about 30% of the total polysaccharide by weight, in one embodiment, from about 4% to about 20% of the total polysaccharide by weight, in one embodiment, from about 5% to about 10% of the total polysaccharide by weight. Alternatively the aforementioned proportions represent percentage by mass. In an alternative embodiment, the binding and release from these bioconjugates can be controlled by the degree of polysaccharide sulfation and by the extent of sulfated polysaccharide sulfate incorporation into the delivery system.

Bioconjugates

In one embodiment, a “bioconjugate” is a molecular complex formed by stably linking two molecules, at least one of which has a biological activity. In one embodiment, a polypeptide of the invention may be operably linked to a sulfated polysaccharide of the invention to form a bioconjugate. In one embodiment, a polypeptide of the invention may be operably linked to alginate sulfate to form a bioconjugate. In another embodiment, a polypeptide of the invention may be operably linked to hyaluronan sulfate to form a bioconjugate.

In one embodiment, the term “operably linked” refers to a juxtaposition wherein the components so described are in a relationship permitting them to function in their intended manner

In one embodiment, the linkage of the bioconjugate, between the sulfated polysaccharide and the polypeptide of interest, is non-covalent. In embodiment, the non-covalent linkage is an ionic bond, an electrostatic interaction, a hydrophobic interaction, or a hydrogen bond or a van der Waals force. In one embodiment, the polypeptide is affinity-bound to the sulfated polysaccharide of the present invention.

In one embodiment, a polypeptide of the invention may be capable of associating with the polysaccharide of the invention to form a bioconjugate. In one embodiment, a polypeptide of the invention may be capable of associating with alginate sulfate to form a bioconjugate. In another embodiment, a polypeptide of the invention may be capable of associating with hyaluronan sulfate to form a bioconjugate. In another embodiment, a polypeptide of the invention may be associated with a sulfated polysaccharide of the invention to form a bioconjugate. In another embodiment, a polypeptide of the invention may be linked to a sulfated polysaccharide of the invention to form a bioconjugate.

In another embodiment, the present invention provides a composition comprising a sulfated hyaluronan bioconjugate. In another embodiment, the present invention provides a method of producing a sulfated hyaluronan bioconjugate comprising providing a preformed scaffold comprising sulfated hyaluronan and hyaluronan and then contacting said scaffold with a polypeptide as described herein.

In another embodiment, the present invention provides a composition comprising a sulfated alginate bioconjugate. In another embodiment, the present invention provides a method of producing a sulfated alginate bioconjugate comprising providing a preformed scaffold comprising sulfated alginate and alginate and then contacting said scaffold with a polypeptide as described herein.

Sulfating polysaccharides endows them with properties which allow binding and controlled release of important signal proteins such as various cytokines and growth factors. Alginate sulfate and hyaluronan sulfate were both found to mimic the biological specificities of heparan sulfate and heparin when forming bioconjugates (see e.g. WO 2007/043050, which is hereby incorporated by reference in its entirety).

In one embodiment, a bioactive polypeptide having a positive charge may be reversibly and non-covalently bound to a sulfated polysaccharide, which carries a negative charge due to its sulfur group.

The methods of manufacturing sulfated polysaccharide bioconjugates have been described (see e.g. U.S. Pat. Pub. 2015/0051148, which is hereby incorporated by reference in its entirety).

Nanoparticles

In one embodiment, compositions of the present invention comprise nanoparticles. Any suitable nanoparticle size can be used. The diameter of the nanoparticle may range between about 0.1 nm and about 500 nm. In one embodiment, the diameter of nanoparticle is about 0.1, 0.5, 1, 10, 50, 100, 150, 200, or 500 nm.

In one embodiment, the present invention provides a nanoparticle comprising alginate sulfate and EGF. In another embodiment, the present invention provides a nanoparticle comprising alginate sulfate and bFGF. In another embodiment, the present invention provides a nanoparticle comprising alginate sulfate, EGF, and bFGF.

In one embodiment, the bioconjugate formed by the polypeptide and polysaccharide forms a nanoparticle. In one embodiment, the nanoparticle is between 1 and 1000 nm in diameter. In another embodiment, the nanoparticle is between 1 and 100 nm in diameter. In another embodiment, the nanoparticle is between 500 and 1000 nm in diameter. In another embodiment, the nanoparticle is between 1 and 500 nm in diameter. In another embodiment, the nanoparticle is between 20 and 70 nm in diameter. In another embodiment, the nanoparticle is between 100 and 200 nm in diameter.

In a particular embodiment, the molecule in the form of nanoparticle is encapsulated by a polysaccharide of the invention.

In one embodiment, the present invention provides a composition comprising a sulfated hyaluronan nanoparticle. In another embodiment, the present invention provides a method of producing a sulfated hyaluronan nanoparticle comprising providing sulfated hyaluronan and a polypeptide as described herein in solution. In one embodiment, the method of producing a sulfated hyaluronan comprises a) replacement of sodium ions of hyaluronan (HA) with hydrogens b) titration with an amine such as a tertiary amine, for example, tributylamine (TBA); and c) O-sulfation of HA-TBA salt with carbodiimide (DCC) and sulfuric acid. In one embodiment, the nanoparticle may then be contacted with an alginate solution and a cross-linker, in vitro or in vivo, thereby forming a matrix comprising the bioconjugate nanoparticle, as described herein.

In one embodiment, the present invention provides a composition comprising a sulfated alginate nanoparticle. In another embodiment, the present invention provides a method of producing a sulfated alginate nanoparticle comprising providing a sulfated alginate and a polypeptide as described herein in solution. In one embodiment, the method of producing a sulfated alginate comprises a) converting sodium alginate to alginic acid by batch ion exchange b) titration with an amine such as a tertiary amine, for example, tributylamine (TBA), yielding alginate-TBA; and c) O-sulfation of the amine salt of alginate by treatment with sulfuric acid and addition of a N,N′-carbodiimide, e.g., N5N′-dicyclohexylcarbodiimide (DCC). In one embodiment, the nanoparticle may then be contacted with an alginate solution and a cross-linker, in vitro or in vivo, thereby forming a matrix comprising the bioconjugate nanoparticle, as described herein.

Linkers

In another embodiment, the polypeptide of the invention is linked to the polysaccharide of the invention via a linker. In one embodiment, alginate is linked to a polypeptide of the present invention via a linker. In one embodiment, the linker is a peptide, wherein the first polypeptide is linked to the polysaccharide via a peptide, which serves as a linker. In one embodiment, the linker is a heparin binding peptide (HBP). In another embodiment, the linker is an amino acid sequence that is enriched in positively-charged amino acids. In one embodiment, the linker is a cell-penetrating peptide (CPP). In one embodiment, CPPs have an amino acid composition that contains a high relative abundance of positively charged amino acids such as lysine or arginine.

In another embodiment, the linker is a linker suitable for binding a negatively charged alginate such as sulfated alginate with a negatively charged molecule (e.g., IL-1RA). In one embodiment, the linker is a HBP. In one embodiment, HBP or other peptides are bound to the alginate by the mechanism described in Sapir et al., 2011, Biomaterials, vol. 32, pages 1838-1847 and Bondalapati et al. Macromol Rapid Commun. 2014 Sep. 15, both incorporated herein by reference.

Hydrogels and Scaffolds

Compositions of the present invention may be in any suitable form, for example, a solid, a semi-solid, or a liquid form. The form may be in any form appropriate to the mode of delivery, for example, hydrogel, beads, implants, microspheres (microbeads), hydrogel microcapsules, sponges, scaffolds, meshes, foams, colloidal dispersions, nanoparticles, and suspensions.

In one embodiment, the composition may be in the form of a hydrogel. The term “hydrogel” as used herein may refer to a network of natural or synthetic hydrophilic polymer chains able to contain water. Examples of compounds able to form such networks are alginate, a partially calcium cross-linked alginate solution, chitosan and viscous hyaluronan. In one embodiment, the bioconjugate of the present invention may be in the form of a flowable gel.

In one embodiment, the composition may be in the form of a scaffold, which in one embodiment, is a pre-formed scaffold. The term “scaffold” as used herein refers to any synthetic or organic structure comprising a void. Non-limiting examples of such scaffolds are molds, casts and voids in damaged tissue in a mammal In one embodiment, the scaffold is a macroporous scaffold. In one embodiment, the macroporous scaffold has pore sizes in the micron range, which in one embodiment, is 50-100 μm. In one embodiment, the scaffold may be a multi-compartment scaffold. In one embodiment, the scaffold is not hydrated until just prior to use. In one embodiment, the scaffold is highly porous (>90%). In one embodiment, the scaffold has an interconnected pore structure. In one embodiment, the pore size is 70-100 μm.

Thus, in one embodiment, alginate may be used to make hydrogel compositions or macroporous scaffolds, whether single or multi-compartment compositions of the present invention. In one embodiment, said hydrogels and scaffolds may comprise bioactive peptides, additives, biological material, or a combination thereof as described herein.

Multi-compartment Hydrogels and Matrices

The composition may be a single compartment composition or a multi-compartment composition. In one embodiment, the composition is a multi-compartment hydrogel or scaffold. In one embodiment, the multi-compartment composition may comprise a plurality of layers. For example, the multi-compartment composition may comprise a first compartment and a second compartment, wherein the first compartment is a first layer and the second compartment is a second layer. In one embodiment, each compartment may include a distinct polypeptide or a combination of polypeptides. In another embodiment, each compartment may include the same polypeptide(s). In one embodiment, one or more compartments or layers may lack a polypeptide.

In one embodiment, a neuroprotective polypeptide is present in a first compartment and an angiogenic polypeptide is present in a second compartment of said multi-compartment composition. In another embodiment, Epidermal Growth Factor (EGF) is present in a first compartment and a basic Fibroblast Growth factor (bFGF) is present in a second compartment of said multi-compartment composition. In yet another embodiment, Epidermal Growth Factor (EGF), basic Fibroblast Growth factor (bFGF), or a combination thereof are present in a first compartment and an additional polypeptide is present in a second compartment of said multi-compartment composition.

In one embodiment, the present invention provides a multi-compartment hydrogel or matrix comprising a first compartment or layer comprising alginate sulfate and EGF and a second compartment or layer comprising alginate sulfate and bFGF. In one embodiment, the multi-compartment hydrogel or matrix comprises alginate. In another embodiment, the multi-compartment hydrogel or matrix comprises alginate sulfate. In another embodiment, the multi-compartment hydrogel or matrix comprises a mixture of alginate and alginate sulfate.

In one embodiment, each compartment of the multi-compartment hydrogel or scaffold has distinct properties. In one embodiment, each compartment has distinct physical properties. In one embodiment, each compartment of the multi-compartment hydrogel or scaffold is in intimal contact with at least one other compartment of the hydrogel.

In one embodiment, one compartment of the multi-compartment hydrogel or scaffold has larger pores, while a second compartment of the multi-compartment hydrogel or scaffold has smaller pores. In one embodiment, the first hydrogel layer has pores in the micron range, while the second hydrogel layer has pores in the submicron range.

In one embodiment, a hydrogel layer has pores in the micron range. In one embodiment, a hydrogel layer has pores of 50-200 μm. In another embodiment, a hydrogel layer has pores of 50-100 μm. In another embodiment, the pores are obtained using ACP, beads, etc. In another embodiment, the pores are 80-120 μm, in another embodiment, 150-200 μm and in another embodiment 250-300 μm.

In one embodiment, a hydrogel as described herein has a pore size that is similar to that of natural extracellular matrix (ECM) in the tissue of interest. In one embodiment, one of the hydrogel layers has pores in the submicron range. In one embodiment, the pores are 3-7 nm. In another embodiment, the pores are 1-10 nm. In another embodiment, the pores are 5-20 nm. In another embodiment, the pores are approximately 6 nm. In another embodiment, the hydrogel has a pore size of 1-1000 nm. In another embodiment, the hydrogel has a pore size of 500-1000 nm. In another embodiment, the hydrogel has a pore size of 5-500 nm. In another embodiment, the hydrogel has a pore size of, 1-500 nm. In another embodiment, the hydrogel has a pore size of 250-750 nm. In another embodiment, the hydrogel has a pore size of 1-250 nm. In another embodiment, the hydrogel has a pore size of 1-100 nm.

In one embodiment, one hydrogel layer will comprise a bioactive polypeptide, while a second hydrogel layer will not comprise a bioactive polypeptide. In one embodiment, one or both layers will comprise additives and autologous biological fluids. In one embodiment, one hydrogel or scaffold layer does not comprise a sulfated polysaccharide. In one embodiment, a second hydrogel or scaffold layer does comprise a sulfated polysaccharide. In one embodiment, the layer that does not comprise a sulfated polysaccharide also does not comprise a bioactive polypeptide. In one embodiment, the layer that comprises a sulfated polysaccharide also comprises a bioactive polypeptide.

In another embodiment, the present invention provides a macroporous scaffold. In one embodiment, a macroporous scaffold as described herein has pores in the micron range, which in one embodiment, is a pore size of 50-100 μm. In another embodiment, the scaffold has a pore size of 1-100 μm. In another embodiment, the scaffold has a pore size of 50-200 μm. In another embodiment, the scaffold has a pore size of 1-50 μm. In another embodiment, the scaffold has a pore size of 25-75 μm. In another embodiment, the scaffold has a pore size of 1-25 μm. In another embodiment, the scaffold has a pore size of 1-10 μm.

In another embodiment, each compartment of the multi-compartment hydrogel or scaffold has distinct chemical properties. In one embodiment, one compartment has a higher concentration or amount of sulfated alginate than a second compartment of the multi-compartment hydrogel or scaffold. In one embodiment, the % w/v of sulfated alginate is 0.6%-0.7% in one layer and 0.8%-0.9% in a second layer. In another embodiment, the % w/v of sulfated alginate is 0.65%-0.75% in one layer and 0.85%-0.95% in a second layer. In another embodiment, the % w/v of sulfated alginate is 0.4%-0.7% in one layer and 0.8%-1.2% in a second layer. In one embodiment, the % w/v of sulfated alginate is 0.67% in one layer and 0.87% in a second layer.

In another embodiment, one compartment has a higher concentration or amount of polypeptide than a second compartment of the multi-compartment hydrogel or scaffold. In another embodiment, one compartment has a higher concentration or amount of EGF and/or bFGF than a second compartment of the multi-compartment hydrogel or scaffold. In another embodiment, one compartment has a higher concentration or amount of EGF than a second compartment of the multi-compartment hydrogel or scaffold. In another embodiment, one compartment has a higher concentration or amount of bFGF than a second compartment of the multi-compartment hydrogel or scaffold. In one embodiment, at least one of the hydrogel layers lacks a polypeptide.

In another embodiment, the different compartments have both distinct physical properties and distinct chemical properties.

Hydrogel Additives

In one embodiment, the composition may further comprise a non-biological additive, which in one embodiment, is neither a cell nor a polypeptide. Examples of an additive include, for example, but are not limited to, hydroxyapatite, calcium phosphate, one or more mannitol beads, one or more magnesium minerals, or a combination thereof.

In one embodiment, methods for including magnesium in alginate gels are known in the art (e.g. WO 2005039662 A2, which is incorporated herein by reference). In one embodiment, magnesium may be used instead of calcium to cross-link a polysaccharide. In another embodiment, both magnesium and calcium may be included as cross-linking agents.

In one embodiment, hydroxyapatite, mannitol beads and calcium phosphate create a porous scaffold. In one embodiment, calcium phosphate serves a dual purpose—i.e., to create a more porous scaffold and to cross-link alginate.

In one embodiment, the present invention comprises compositions comprising a biological additive. In one embodiment, the biological additive is a biological fluid or biological material. Examples of a biological fluid or biological material include, for example, but are not limited to, platelet rich plasma (PRP), bone marrow aspirate, and serum. In another embodiment, the biological material is a cell. In one embodiment, the biological material is autologous to the person whom is receiving or will be administered the multi-compartment hydrogel or scaffold. In another embodiment, the biological material is heterologous to the person whom is receiving or will be administered the multi-compartment hydrogel or scaffold. In one embodiment, the biological material is platelet rich plasma (PRP), bone marrow aspirate, serum or a combination thereof. In another embodiment, the biological material is autologous platelet rich plasma (PRP), autologous bone marrow aspirate, autologous serum or a combination thereof.

In one embodiment, the biological fluid or material is an autologous fluid or material. In another embodiment, the biological fluid or material is a heterologous fluid or material. In another embodiment, the biological fluid or material is an allogenic fluid or material. Accordingly, in one embodiment, the composition may further comprise a biological fluid or material from a patient in need of said composition for regenerating or repairing a damaged tissue in the patient, such as spinal cord tissue.

In one embodiment, methods of harvesting, processing, activation, and administering PRP are known in the art (e.g. Sakata et al. Tissue Eng Part B Rev. 2015 October; 21 (5):461-73. doi: 10.1089/ten.TEB.2014.0661. Epub 2015 Jul. 14, incorporated herein by reference in its entirety, page 465 column 2). In one embodiment, methods used to prepare PRP concentrate the platelets in some amount of autologous serum, induce the platelets to release their morphogens prior to administration, whether by chemical or by physical stimulus ex vivo, or a combination thereof.

In one embodiment, compositions of and for use in the methods of the present invention lack a progenitor cell. In another embodiment, compositions of and for use in the methods of the present invention comprise a progenitor cell. In one embodiment, the progenitor cell is a neural progenitor cell (NPC). In another embodiment, the progenitor cell is a stem cell. In one embodiment, the stem cell is seeded into the composition prior to administration of the composition to a subject. In one embodiment, the stem cell is a mesenchymal stem cell. In one embodiment, the cell is a tissue precursor cell. In one embodiment, the stem cell is a neural stem cell. In one embodiment, the stem cell is an autologous stem cell derived from a subject in need of a treatment. In another embodiment, the stem cell is a non-autologous or heterologous stem cell derived from an individual different from a subject in need of a treatment.

Supporting Matrix

In some embodiments, the composition may further comprise a supporting matrix. The matrix may serve as support or as a carrier for the bioconjugate and may be made up of particles or porous materials. The matrix material may be flexible and amenable to be fixed in place preventing its migration to an unintended location. In one embodiment, the supporting matrix comprises a polymer selected from the group consisting of a polysaccharide, a protein, an extracellular matrix component, and a synthetic polymer, or a mixture thereof. In one embodiment, the polymer is alginate.

In one embodiment, the extracellular matrix component may be a proteoglycan, such as heparan sulfate, chondroitin sulfate, keratan sulfate. In another embodiment, the extracellular matrix component may be a non-proteoglycan polysaccharide, such as hyaluronic acid. In another embodiment, the extracellular matrix component may be a fiber, which in one embodiment, is collagen or elastin. In another embodiment, the extracellular matrix component may be fibronectin or laminin.

In one embodiment, the synthetic polymer is selected from the group consisting of: PBT (Polybutylene terephthalate), PCL (Poly(ε-caprolactone)), PDLLA (Poly(DL-lactide)), PEE: (Poly(ether ester)), PEG (Poly(ethylene glycol)), PEO (Poly(ethylene oxide)), PGA (Polyglycolide), PLA (Polylactide), PLGA (Poly(lactic acid-glycolic acid)), PPF (Poly(propylene fumarate)), and PVA (Polyvinyl alcohol), or a combination thereof.

In one embodiment, the structure of a hydrogel of the present invention mimics the structure of natural extracellular matrix.

In one embodiment, the polysaccharides in the multi-compartment hydrogel or scaffold are cross-linked by calcium. In one embodiment, the polysaccharides in the multi-compartment hydrogel or scaffold are cross-linked using calcium phosphate, calcium chloride, calcium gluconate or a combination thereof. In one embodiment, the calcium cross-linked polysaccharide is in the form of solid macroporous scaffold.

In one embodiment, a composition of the present invention is injectable, biodegradable, bioerodable, or a combination thereof. In one embodiment, the composition of the present invention is biodegradable. In another embodiment, a composition of the present invention is bioerodable. In one embodiment, a composition of the present invention comprising alginate undergoes dissolution and erosion in the body of a mammal. In another embodiment, the composition of the present invention does not elicit an immune response in a subject. In one embodiment, the composition of the present invention has a half-life of between one and two months.

In one embodiment, the composition is homogenous or isotropic. In another embodiment, the composition is denser or less porous in one or more surface portions than its core. In yet another embodiment, the composition is denser or less porous in one or more predetermined portions.

The composition of the invention may be administered via any suitable method known to one of skilled in the art. Examples of such method include, but is not limited to, intraliver, intradermal, transdermal (e.g. in slow release formulations), intramuscular, intraperitoneal, intravenous, intracoronary, subcutaneous, oral, epidural, topical, and intranasal routes.

The administration of the invention also encompasses surgically administering, implanting, inserting, or injecting the implant (or sections thereof) into a subject. The implant (or section) can be located subcutaneously, intramuscularly, or located at another body location that allows the implant to perform its intended function. Generally, implants (or sections) are administered by subcutaneous implantation at sites including, but not limited to, the upper arm, back, or abdomen of a subject. Other suitable sites for administration may be readily determined by a medical professional. Multiple implants or sections may be administered to achieve a desired dosage for treatment. Any other therapeutically efficacious route of administration can be used. Administration may also include systemic or local administration of the composition of the invention.

The present invention further contemplates adding a pharmaceutically acceptable carrier to the sulfated polysaccharide-bioactive polypeptide bioconjugate. The term “pharmaceutically acceptable carrier” refers to a vehicle which delivers the active components to the intended target and which will not cause harm to humans or other recipient organisms. As used herein, “pharmaceutical” will be understood to encompass both human and veterinary pharmaceuticals. Useful carriers include, for example, water, acetone, ethanol, ethylene glycol, propylene glycol, butane-1, 3-diol, isopropyl myristate, isopropyl palmitate, mineral oil and polymers composed of chemical substances like polyglycolic acid or polyhydroxybutyrate or natural polymers like collagen, fibrin or polysaccharides like chitosan and alginate. The carrier may be in any form appropriate to the mode of delivery, for example, solutions, colloidal dispersions, emulsions (oil-in-water or water-in-oil), suspensions, creams, lotions, gels, foams, mousses, sprays and the like. Methodology and components for formulation of pharmaceutical compositions are well known and can be found, for example, in Remington's Pharmaceutical Sciences, Eighteenth Edition, A. R. Gennaro, Ed., Mack Publishing Co. Easton Pa., 1990. In one embodiment of the invention, the carrier is an aqueous buffer. In another embodiment, the carrier is a polysaccharide and is in one embodiment, alginate hydrogel or in another embodiment, hyaluronic acid.

Uses and Methods of Treatment

Examples of a disease or disorder treated by the composition of the invention include, but are not limited to, a spinal cord injury, damage to spinal cord tissue, and a disease or disorder associated with spinal cord. Examples of a disease or disorder associated with spinal cord include, but not limited to, arachnoditis, arterial venous malformation, brown sequard syndrome, cauda equina syndrome, cerebral palsy, central cord syndrome, Guillian-Barre syndrome, back and spine conditions, multiple sclerosis, polio, spinal cord tumor, spina bifida, spinal stenosis, stroke, syringomyelia, and transverse myelitis.

In a particular example, the composition of the invention is used for repairing or regenerating a damaged spinal cord tissue.

In one embodiment, the methods of the present invention may be used to treat a disease, condition or disorder described herein. In another embodiment, the methods of the present invention may be used to prevent a disease, condition or disorder described herein. In another embodiment, the methods of the present invention may be used to suppress a disease, condition or disorder described herein. In another embodiment, the methods of the present invention may be used to inhibit a disease, condition or disorder described herein.

In one embodiment, “treating” as used herein refers to therapeutic treatment. In one embodiment, “preventing”, “suppressing” or “inhibiting” as used herein refers to prophylactic or preventative measures, wherein the object is to prevent or lessen the targeted condition or disorder as described herein. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

The composition of the invention can also be used for enhancing an immunotolerant response. Examples of immunotolerant responses include, but are not limited to, allograft success, lack of allograft rejection, suppression of autoimmune disorder, suppression of an immune response to an allocell transplantation, suppression of allocell apoptosis, an increase in allocell survival, stimulation of vascularization of allocell transplant, prolonged presentation of said bioactive polypeptide, suppression of inflammatory signaling, suppression of dendritic cell maturation, suppression of CD8+ T cell cytotoxicity response, and stimulation of regulatory T cell differentiation.

Treatment with Sulfated Polysaccharides

In another embodiment, the invention provides a method for repairing or regenerating damaged spinal cord tissue in a subject, the method comprising the step of: administering to said subject a composition comprising a polysaccharide and a polypeptide operably linked to said polysaccharide, wherein said polysaccharide is a sulfated alginate, or sulfated hyaluronan, and wherein said polypeptide is a growth factor, thereby repairing or regenerating said damaged spinal cord tissue in said subject.

In another embodiment, the invention provides a method for treating a spinal cord injury in a subject, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is a growth factor, thereby treating said spinal cord injury in said subject.

In another embodiment, the invention provides a method for promoting the regrowth of one or more axons in a subject with a spinal cord injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is a growth factor, thereby promoting the regrowth of said axons in said subject.

In one embodiment, the axons are corticospinal tract (CST) axons. In one embodiment, the CST axons are motor CST axons. In one embodiment, the axons are positively labeled using biotinylated dextran amines (BDA).

In another embodiment, the invention provides a method for increasing the number of surviving neurons, sensory fibres, or a combination thereof in a subject with a spinal injury, the method comprising the step of: administering to said subject a composition comprising a polysaccharide and a polypeptide linked to said polysaccharide, wherein said polysaccharide is a sulfated alginate or sulfated hyaluronan, and wherein said polypeptide is a growth factor, thereby increasing the number of surviving neurons, sensory fibres, or a combination thereof in said subject.

In one embodiment, the neurons are choline acetyltransferase positive motoneurons.

In another embodiment, the invention provides a method for increasing the number of blood vessels in a central lesion in a subject with a spinal injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is a growth factor, thereby increasing the number of blood vessels in said subject with a spinal injury.

In one embodiment, the vascular supply to the tissue of said spinal cord is preserved. In one embodiment, the subject does not develop central sensitization, which, in one embodiment, is hyperalgesia, allodynia, or a combination thereof.

In another embodiment, the invention provides a method for repairing or regenerating damaged spinal cord tissue in a subject, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is an epidermal growth factor (EGF), a fibroblast growth factor (FGF), or a combination thereof, thereby repairing or regenerating said damaged spinal cord tissue in said subject.

In another embodiment, the invention provides a method for treating a spinal cord injury in a subject, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is an epidermal growth factor (EGF), a fibroblast growth factor (FGF), or a combination thereof, thereby treating said spinal cord injury in said subject.

In another embodiment, the invention provides a method for promoting the regrowth of one or more axons in a subject with a spinal cord injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is an epidermal growth factor (EGF), a fibroblast growth factor (FGF), or a combination thereof, thereby promoting the regrowth of said axons in said subject.

In another embodiment, the invention provides a method for increasing the number of surviving neurons, sensory fibres, or a combination thereof in a subject with a spinal injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is an epidermal growth factor (EGF), a fibroblast growth factor (FGF), or a combination thereof, thereby increasing the number of surviving neurons, sensory fibres, or a combination thereof in said subject.

In another embodiment, the invention provides a method for increasing the number of blood vessels in a central lesion in a subject with a spinal injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is an epidermal growth factor (EGF), a fibroblast growth factor (FGF), or a combination thereof, thereby increasing the number of blood vessels in said subject with a spinal injury.

In another embodiment, the present invention provides a composition for use in repairing or regenerating a damaged spinal cord tissue in a subject, the composition comprising: a polysaccharide and a polypeptide linked to said polysaccharide, wherein said polysaccharide is a sulfated alginate or sulfated hyaluronan, and wherein said polypeptide is a growth factor.

In another embodiment, the present invention provides a composition for use in treating a spinal cord injury in a subject, the composition comprising: a polysaccharide and a polypeptide linked to said polysaccharide, wherein said polysaccharide is a sulfated alginate or sulfated hyaluronan, and wherein said polypeptide is a growth factor.

In another embodiment, the present invention provides a composition for use in promoting the regrowth of one or more axons in a subject with a spinal cord injury, the composition comprising: a polysaccharide and a polypeptide linked to said polysaccharide, wherein said polysaccharide is a sulfated alginate or sulfated hyaluronan, and wherein said polypeptide is a growth factor.

In another embodiment, the present invention provides a composition for use in increasing the number of surviving neurons, sensory fibres, or a combination thereof in a subject with a spinal injury, the composition comprising: a polysaccharide and a polypeptide linked to said polysaccharide, wherein said polysaccharide is a sulfated alginate or sulfated hyaluronan, and wherein said polypeptide is a growth factor.

In another embodiment, the present invention provides a composition for use in increasing the number of blood vessels in a central lesion in a subject with a spinal injury, the composition comprising: a polysaccharide and a polypeptide linked to said polysaccharide, wherein said polysaccharide is a sulfated alginate or sulfated hyaluronan, and wherein said polypeptide is a growth factor.

In another embodiment, the present invention provides for a use of a composition comprising: a polysaccharide and a polypeptide linked to said polysaccharide, wherein said polysaccharide is a sulfated alginate or sulfated hyaluronan, and wherein said polypeptide is a growth factor in the preparation of a composition for use in repairing or regenerating a damaged spinal cord tissue in a subject.

In another embodiment, the present invention provides for a use of a composition comprising: a polysaccharide and a polypeptide linked to said polysaccharide, wherein said polysaccharide is a sulfated alginate or sulfated hyaluronan, and wherein said polypeptide is a growth factor for use in treating a spinal cord injury in a subject.

In another embodiment, the present invention provides for a use of a composition comprising: a polysaccharide and a polypeptide linked to said polysaccharide, wherein said polysaccharide is a sulfated alginate or sulfated hyaluronan, and wherein said polypeptide is a growth factor for promoting the regrowth of one or more axons in a subject with a spinal cord injury.

In another embodiment, the present invention provides for a use of a composition comprising: a polysaccharide and a polypeptide linked to said polysaccharide, wherein said polysaccharide is a sulfated alginate or sulfated hyaluronan, and wherein said polypeptide is a growth factor for increasing the number of surviving neurons, sensory fibres, or a combination thereof in a subject with a spinal injury.

In another embodiment, the present invention provides for a use of a composition comprising: a polysaccharide and a polypeptide linked to said polysaccharide, wherein said polysaccharide is a sulfated alginate or sulfated hyaluronan, and wherein said polypeptide is a growth factor for increasing the number of blood vessels in a central lesion in a subject with a spinal injury.

Treatment with Sulfated Polysaccharides Lacking a Bioactive Polypeptide

In another embodiment, the invention provides a method for repairing or regenerating damaged spinal cord tissue in a subject, the method comprising the step of: administering to said subject a composition comprising a sulfated polysaccharide lacking a bioactive polypeptide, thereby repairing or regenerating said damaged spinal cord tissue in said subject.

In another embodiment, the invention provides a method for treating a spinal cord injury in a subject, the method comprising the step of: administering to said subject a composition comprising a sulfated polysaccharide lacking a bioactive polypeptide, thereby treating said spinal cord injury in said subject.

In another embodiment, the invention provides a method for promoting the regrowth of one or more axons in a subject with a spinal cord injury, the method comprising the step of: administering to said subject a composition comprising a sulfated polysaccharide lacking a bioactive polypeptide, thereby promoting the regrowth of said axons in said subject.

In another embodiment, the invention provides a method for increasing the number of surviving neurons, sensory fibres, or a combination thereof in a subject with a spinal injury, the method comprising the step of: administering to said subject a composition comprising a sulfated polysaccharide lacking a bioactive polypeptide, thereby increasing the number of surviving neurons, sensory fibres, or a combination thereof in said subject.

In another embodiment, the invention provides a method for increasing the number of blood vessels in a central lesion in a subject with a spinal injury, the method comprising the step of: administering to said subject a composition comprising a sulfated polysaccharide lacking a bioactive polypeptide, thereby increasing the number of blood vessels in said subject with a spinal injury.

In one embodiment, said sulfated polysaccharide is sulfated alginate, sulfated hyaluronan, or a combination thereof.

In one embodiment, the method comprises the step of administering a matrix comprising the composition as described herein. In one embodiment the matrix is a hydrogel. In one embodiment, said matrix comprises a polymer selected from the group consisting of a polysaccharide, a protein, an extracellular matrix component, a synthetic polymer, and a mixture thereof. In one embodiment, the matrix is an alginate matrix. Therefore, in one embodiment, the present invention provides methods comprising the step of administering an alginate hydrogel composition comprising alginate sulfate, sulfated hyaluronan, or a combination thereof. In one embodiment, the alginate hydrogel composition lacks a bioactive polypeptide. In one embodiment, the sulfated polysaccharide recruits and/or binds endogenous bioactive polypeptides and releases them slowly at the site of injury.

Treatment with Non-sulfated Alginate

In another embodiment, the invention provides a method for repairing or regenerating damaged spinal cord tissue in a subject, the method comprising the step of: administering to said subject a composition comprising a polysaccharide and a polypeptide operably linked to said polysaccharide, wherein said polysaccharide is an alginate, and wherein said polypeptide is a growth factor, thereby repairing or regenerating said damaged spinal cord tissue in said subject.

In another embodiment, the invention provides a method for treating a spinal cord injury in a subject, the method comprising the step of: administering to said subject a composition comprising a polysaccharide and a polypeptide operably linked to said polysaccharide, wherein said polysaccharide is an alginate, and wherein said polypeptide is a growth factor, thereby treating said spinal cord injury in said subject.

In another embodiment, the invention provides a method for promoting the regrowth of one or more axons in a subject with a spinal cord injury, the method comprising the step of: administering to said subject a composition comprising a polysaccharide and a polypeptide operably linked to said polysaccharide, wherein said polysaccharide is an alginate, and wherein said polypeptide is a growth factor, thereby promoting the regrowth of said axons in said subject.

In one embodiment, the axons are corticospinal tract (CST) axons. In one embodiment, the CST axons are motor CST axons. In one embodiment, the axons are positively labeled using biotinylated dextran amines (BDA).

In another embodiment, the invention provides a method for increasing the number of surviving neurons, sensory fibres, or a combination thereof in a subject with a spinal injury, the method comprising the step of: administering to said subject a composition comprising a polysaccharide and a polypeptide linked to said polysaccharide, wherein said polysaccharide is an alginate, and wherein said polypeptide is a growth factor, thereby increasing the number of surviving neurons, sensory fibres, or a combination thereof in said subject. In one embodiment, the neurons are choline acetyltransferase positive motoneurons.

In another embodiment, the invention provides a method for increasing the number of blood vessels in a central lesion in a subject with a spinal injury, the method comprising the step of: administering to said subject a composition comprising a polysaccharide and a polypeptide operably linked to said polysaccharide, wherein said polysaccharide is an alginate, and wherein said polypeptide is a growth factor, thereby increasing the number of blood vessels in said subject with a spinal injury.

In one embodiment, the vascular supply to the tissue of said spinal cord is preserved. In one embodiment, the subject does not develop central sensitization, which, in one embodiment, is hyperalgesia, allodynia, or a combination thereof.

In another embodiment, the invention provides a method for repairing or regenerating damaged spinal cord tissue in a subject, the method comprising the step of: administering to said subject a composition comprising a polysaccharide and a polypeptide operably linked to said polysaccharide, wherein said polysaccharide is an alginate, and wherein said polypeptide is an epidermal growth factor (EGF), a fibroblast growth factor (FGF), or a combination thereof, thereby repairing or regenerating said damaged spinal cord tissue in said subject.

In another embodiment, the invention provides a method for treating a spinal cord injury in a subject, the method comprising the step of: administering to said subject a composition comprising a polysaccharide and a polypeptide operably linked to said polysaccharide, wherein said polysaccharide is an alginate, and wherein said polypeptide is an epidermal growth factor (EGF), a fibroblast growth factor (FGF), or a combination thereof, thereby treating said spinal cord injury in said subject.

In another embodiment, the invention provides a method for promoting the regrowth of one or more axons in a subject with a spinal cord injury, the method comprising the step of: administering to said subject a composition comprising a polysaccharide and a polypeptide operably linked to said polysaccharide, wherein said polysaccharide is an alginate, and wherein said polypeptide is an epidermal growth factor (EGF), a fibroblast growth factor (FGF), or a combination thereof, thereby promoting the regrowth of said axons in said subject.

In another embodiment, the invention provides a method for increasing the number of surviving neurons, sensory fibres, or a combination thereof in a subject with a spinal injury, the method comprising the step of: administering to said subject a composition comprising a polysaccharide and a polypeptide operably linked to said polysaccharide, wherein said polysaccharide is an alginate, and wherein said polypeptide is an epidermal growth factor (EGF), a fibroblast growth factor (FGF), or a combination thereof, thereby increasing the number of surviving neurons, sensory fibres, or a combination thereof in said subject.

In another embodiment, the invention provides a method for increasing the number of blood vessels in a central lesion in a subject with a spinal injury, the method comprising the step of: administering to said subject a composition comprising a polysaccharide and a polypeptide operably linked to said polysaccharide, wherein said polysaccharide is an alginate, and wherein said polypeptide is an epidermal growth factor (EGF), a fibroblast growth factor (FGF), or a combination thereof, thereby increasing the number of blood vessels in said subject with a spinal injury.

In one embodiment, EGF, FGF, or both are linked to alginate via a linker. In one embodiment, the linkage is covalent.

In another embodiment, the present invention provides a composition for use in repairing or regenerating a damaged spinal cord tissue in a subject, the composition comprising: a polysaccharide and a polypeptide linked to said polysaccharide, wherein said polysaccharide is an alginate, and wherein said polypeptide is a growth factor.

In another embodiment, the present invention provides a composition for use in treating a spinal cord injury in a subject, the composition comprising: a polysaccharide and a polypeptide linked to said polysaccharide, wherein said polysaccharide is an alginate, and wherein said polypeptide is a growth factor.

In another embodiment, the present invention provides a composition for use in promoting the regrowth of one or more axons in a subject with a spinal cord injury, the composition comprising: a polysaccharide and a polypeptide linked to said polysaccharide, wherein said polysaccharide is an alginate, and wherein said polypeptide is a growth factor.

In another embodiment, the present invention provides a composition for use in increasing the number of surviving neurons, sensory fibres, or a combination thereof in a subject with a spinal injury, the composition comprising: a polysaccharide and a polypeptide linked to said polysaccharide, wherein said polysaccharide is an alginate, and wherein said polypeptide is a growth factor.

In another embodiment, the present invention provides a composition for use in increasing the number of blood vessels in a central lesion in a subject with a spinal injury, the composition comprising: a polysaccharide and a polypeptide linked to said polysaccharide, wherein said polysaccharide is an alginate, and wherein said polypeptide is a growth factor.

In another embodiment, the present invention provides for a use of a composition comprising: a polysaccharide and a polypeptide linked to said polysaccharide, wherein said polysaccharide is an alginate, and wherein said polypeptide is a growth factor in the preparation of a composition for use in repairing or regenerating a damaged spinal cord tissue in a subject.

In another embodiment, the present invention provides for a use of a composition comprising: a polysaccharide and a polypeptide linked to said polysaccharide, wherein said polysaccharide is an alginate, and wherein said polypeptide is a growth factor for use in treating a spinal cord injury in a subject.

In another embodiment, the present invention provides for a use of a composition comprising: a polysaccharide and a polypeptide linked to said polysaccharide, wherein said polysaccharide is an alginate, and wherein said polypeptide is a growth factor for promoting the regrowth of one or more axons in a subject with a spinal cord injury.

In another embodiment, the present invention provides for a use of a composition comprising: a polysaccharide and a polypeptide linked to said polysaccharide, wherein said polysaccharide is an alginate, and wherein said polypeptide is a growth factor for increasing the number of surviving neurons, sensory fibres, or a combination thereof in a subject with a spinal injury.

In another embodiment, the present invention provides for a use of a composition comprising: a polysaccharide and a polypeptide linked to said polysaccharide, wherein said polysaccharide is an alginate, and wherein said polypeptide is a growth factor for increasing the number of blood vessels in a central lesion in a subject with a spinal injury.

Treatment with Sulfated Alginate Lacking a Bioactive Polypeptide

In another embodiment, the invention provides a method for repairing or regenerating damaged spinal cord tissue in a subject, the method comprising the step of: administering to said subject a composition comprising an alginate sulfate and lacking a bioactive polypeptide, thereby repairing or regenerating said damaged spinal cord tissue in said subject.

In another embodiment, the invention provides a method for treating a spinal cord injury in a subject, the method comprising the step of: administering to said subject a composition comprising an alginate sulfate and lacking a bioactive polypeptide, thereby treating said spinal cord injury in said subject.

In another embodiment, the invention provides a method for promoting the regrowth of one or more axons in a subject with a spinal cord injury, the method comprising the step of: administering to said subject a composition comprising an alginate sulfate and lacking a bioactive polypeptide, thereby promoting the regrowth of said axons in said subject.

In another embodiment, the invention provides a method for increasing the number of surviving neurons, sensory fibres, or a combination thereof in a subject with a spinal injury, the method comprising the step of: administering to said subject a composition comprising an alginate sulfate and lacking a bioactive polypeptide, thereby increasing the number of surviving neurons, sensory fibres, or a combination thereof in said subject.

In another embodiment, the invention provides a method for increasing the number of blood vessels in a central lesion in a subject with a spinal injury, the method comprising the step of: administering to said subject a composition comprising an alginate sulfate and lacking a bioactive polypeptide, thereby increasing the number of blood vessels in said subject with a spinal injury.

In one embodiment, the method comprises the step of administering a matrix comprising the composition as described herein. In one embodiment the matrix is a hydrogel. In one embodiment, said matrix comprises a polymer selected from the group consisting of a polysaccharide, a protein, an extracellular matrix component, a synthetic polymer, and a mixture thereof. In one embodiment, the matrix is an alginate matrix. Therefore, in one embodiment, the present invention provides methods comprising the step of administering an alginate hydrogel composition comprising alginate sulfate. In one embodiment, the alginate hydrogel composition lacks a bioactive polypeptide. In one embodiment, the alginate sulfate recruits and binds endogenous bioactive polypeptides and releases them slowly at the site of injury.

Treatment with Non-sulfated Alginate Lacking a Bioactive Polypeptide

In another embodiment, the invention provides a method for repairing or regenerating damaged spinal cord tissue in a subject, the method comprising the step of: administering to said subject a composition comprising an alginate lacking a bioactive polypeptide, thereby repairing or regenerating said damaged spinal cord tissue in said subject.

In another embodiment, the invention provides a method for treating a spinal cord injury in a subject, the method comprising the step of: administering to said subject a composition comprising an alginate lacking a bioactive polypeptide, thereby treating said spinal cord injury in said subject.

In another embodiment, the invention provides a method for promoting the regrowth of one or more axons in a subject with a spinal cord injury, the method comprising the step of: administering to said subject a composition comprising an alginate lacking a bioactive polypeptide, thereby promoting the regrowth of said axons in said subject.

In another embodiment, the invention provides a method for increasing the number of surviving neurons, sensory fibres, or a combination thereof in a subject with a spinal injury, the method comprising the step of: administering to said subject a composition comprising an alginate lacking a bioactive polypeptide, thereby increasing the number of surviving neurons, sensory fibres, or a combination thereof in said subject.

In another embodiment, the invention provides a method for increasing the number of blood vessels in a central lesion in a subject with a spinal injury, the method comprising the step of: administering to said subject a composition comprising an alginate lacking a bioactive polypeptide, thereby increasing the number of blood vessels in said subject with a spinal injury.

In one embodiment, the present invention provides a method for repairing or regenerating a spinal cord tissue in a subject, the method comprising the step of administering to said subject a composition of the present invention as described herein. In one embodiment, the present invention provides a method for repairing damaged spinal cord tissue in a subject, the method comprising the step of administering to said subject a composition of the present invention as described herein. In one embodiment, the present invention provides a method for regenerating spinal cord tissue in a subject, the method comprising the step of administering to said subject a composition of the present invention as described herein.

In another embodiment, the present invention provides use of a composition of the present invention as described herein for repairing or regenerating a spinal cord tissue in a subject. In another embodiment, the present invention provides use of a composition of the present invention as described herein for repairing damaged spinal cord tissue in a subject. In another embodiment, the present invention provides use of a composition of the present invention as described herein for regenerating spinal cord tissue in a subject.

In another embodiment, the present invention provides use of a composition of the present invention as described herein for treating a spinal cord injury in a subject. In another embodiment, the present invention provides use of a composition of the present invention as described herein for promoting the regrowth of one or more axons in a subject with a spinal cord injury. In another embodiment, the present invention provides use of a composition of the present invention as described herein for increasing the number of surviving neurons, sensory fibres, or a combination thereof in a subject with a spinal injury. In another embodiment, the present invention provides use of a composition of the present invention as described herein for increasing the number of blood vessels in a central lesion in a subject with a spinal injury.

In another embodiment, the present invention provides the use of a composition of the present invention as described herein in the manufacture of a medicament for repairing or regenerating a spinal cord tissue in a subject. In another embodiment, the present invention provides the use of a composition of the present invention as described herein in the manufacture of a medicament for repairing damaged spinal cord tissue in a subject. In another embodiment, the present invention provides the use of a composition of the present invention as described herein in the manufacture of a medicament for regenerating spinal cord tissue in a subject.

In another embodiment, the present invention provides use of a composition of the present invention as described herein in the manufacture of a medicament for treating a spinal cord injury in a subject. In another embodiment, the present invention provides use of a composition of the present invention as described herein in the manufacture of a medicament for promoting the regrowth of one or more axons in a subject with a spinal cord injury. In another embodiment, the present invention provides use of a composition of the present invention as described herein in the manufacture of a medicament for increasing the number of surviving neurons, sensory fibres, or a combination thereof in a subject with a spinal injury. In another embodiment, the present invention provides use of a composition of the present invention as described herein in the manufacture of a medicament for increasing the number of blood vessels in a central lesion in a subject with a spinal injury.

In another embodiment, the present invention provides a method for controlling inflammation after spinal cord injury, the method comprising the step of administering to said subject a composition of the present invention as described herein. In one embodiment, the inflammation is acute inflammation. In one embodiment, the acute inflammation is characterized by fluid accumulation, the recruitment of immune cells, the recruitment of microglia, or a combination thereof. In another embodiment, the inflammation is chronic inflammation.

In another embodiment, the present invention provides a method for promoting microglial/macrophage cell polarization, the method comprising the step of administering to said subject a composition of the present invention as described herein. In one embodiment, the microglial/macrophage cell polarization is promoted in a spinal cord, which, in one embodiment, is an injured spinal cord.

In another embodiment, the present invention provides a method for promoting conversion of M1 pro-inflammatory to M2 reparative macrophages, the method comprising the step of administering to said subject a composition of the present invention as described herein. In one embodiment, the microglial/macrophage cell polarization is promoted in a spinal cord, which, in one embodiment, is an injured spinal cord.

In another embodiment, the methods of the present invention further comprise administering phosphatidylserine (PS) presenting liposomes at the lesion site of the spinal cord injury. In one embodiment, PS-presenting liposomes promote switching of macrophages/microglia phenotype to M2.

In another embodiment, the methods of the present invention comprise administering PS-presenting liposomes. In another embodiment, the methods of the present invention comprise administering a sulfated alginate with or without a growth factor, PS-presenting liposomes.

In one embodiment, the step of administering comprises applying a cross-linking agent to the defect surface prior to applying the first layer of gel. In one embodiment, additional cross linking agent is applied to the first gel layer after it is applied to the defect surface in the spinal cord. In one embodiment, additional cross linking agent is applied to the second gel layer after it is applied to the first gel layer. In one embodiment, the cross-linking agent is calcium chloride.

In one embodiment, the step of administering comprises implanting said composition as part of a solid macroporous preformed scaffold. In one embodiment, a preformed scaffold comprises a polypeptide of interest. In one embodiment, a scaffold is stored dry until use.

In one embodiment, the term “subject” includes, but is not limited to, a human. The methods of treatment described herein can be used to treat any suitable mammal, including primates, such as monkeys and humans, horses, cows, sheep, pigs, goats, cats, dogs, rabbits, birds such as turkey, chickens, and ducks, and rodents such as rats, mice, guinea pigs, and hamsters. In a particular embodiment, the mammal to be treated is human. Other subjects include species that are commonly used in scientific research, animal husbandry or as human companions.

In one embodiment, the methods of the present invention may be used in combination with other known methods for repairing or regenerating damaged spinal cord tissue; treating a spinal cord injury; promoting the regrowth of one or more axons; increasing the number of surviving neurons, sensory fibres, or a combination thereof; or increasing the number of blood vessels in a central lesion. Such methods may include administration of methylprednisolone (A-Methapred, Solu-Medrol); Autologous incubated macrophages (ProCord); GM-1 (Sygen), Gacyclidine (GK-11); Thyrotropin Releasing Hormone; Minocycline (Minocin); erythropoieting (EPO); inosine; Rho antagonists such as Cethrin; Rolipram; ATI-355 (NOGO); chondroitinase; 4-aminopyrideine (Fampridine); Gabapentin, or a combination thereof. In another embodiment, the methods of the present invention may be used in conjunction with transplantation of peripheral nerve bridges; stem cell transplant, such as bone marrow stromal cells, other cell transplant such as embryonic olfactory cortex cells or nasal olfactory ensheathing cells; Schwann cells; progenitor cell transplant; functional electrical stimulation (FES); including phrenic nerve; sacral roots; limb muscles, or a combination thereof.

All patents, patent applications, and scientific publications cited herein are hereby incorporated by reference in their entirety.

The following examples are presented in order to more fully illustrate the preferred embodiments of the invention. They should in no way be construed, however, as limiting the broad scope of the invention.

### EXAMPLES

### Example 1

### Delivery of Alginate Scaffold Releasing Two Trophic Factors for Spinal Cord Injury Repair

Spinal cord injury (SCI) has been implicated in neural cell loss and consequently functional motor and sensory impairment. In this study, we propose that an alginate-based neurobridge enriched with/without trophic growth factors (GFs) can be utilized as a therapeutic approach for spinal cord repair. The bioavailability of key GFs, such as Epidermal Growth factor (EGF) and basic Fibroblast Growth Factor (bFGF) released from injected affinity-binding alginate biomaterial to the central lesion site significantly enhanced the sparing of spinal cord tissue and increased the number of surviving neurons (choline acetyltransferase positive motoneurons) and sensory fibres. In addition, we document an enhanced outgrowth of corticospinal tract axons and the presence of blood vessels at the central lesion. Tissue proteomics performed at 3, 7 and 10 days after SCI in rats indicated the presence of anti-inflammatory factors in segments above the central lesion site, whereas in segments below, neurite outgrowth factors, inflammatory cytokines and chondroitin sulfate proteoglycan of the lectican protein family were overexpressed. Collectively, based on our data, we confirm that functional recovery was significantly improved in SCI groups receiving alginate scaffold with affinity-bound growth factors (ALG+GFs), compared to SCI animals without biomaterial treatment.

In the present study, we performed biomaterial treatment at 7 days after SCI followed by tissue microproteomics and immunocytochemistry. Our microproteomic data based on high-resolution (HR) MS/MS shot-gun procedures and statistical analyses indicated that the caudal region 7-10 days post SCI compared to that of 3 days post SCI is able to initiate neurogenesis if trophic and inflammatory inhibitors factors are present on-site. We therefore addressed the therapeutic efficacy of the affinity-binding alginate scaffold as described herein in terms of functional recovery and nerve tissue repair. Specifically, we investigated its influence on: (i) tissue sparing via reduction of the central cavity and enhanced survival of neuronal populations, ii) neurite outgrowth, iii) angiogenesis, iv) response of astrocytes and microglia involved in inflammation and scarring, and v) functional recovery of sensory-motor pathways during a period of 49 days after SCI in rats.

### Materials and Methods

Experimental Groups.

Male Wistar albino rats weighing 290-320 g were divided into 5 groups: 1) sham-operated SCI group (n=6), 2) sham-operated and SCI rats (ALG+ALG+GFs) after biotinylated dextran amines (BDA) tracing (n=10), 3) SCI group receiving saline injection (SCI+SAL) (n=8), 4) SCI group receiving an injection of alginate scaffold/lacking growths factors (SCI+ALG) (n=8), and 5) SCI group receiving an injection of alginate scaffold with affinity-bound EGF and bFGF (SCI+ALG+GFs) (n=8). During the survival, rats were behaviourally tested and after 49 day post-injury, all groups were sacrificed and spinal cord tissue was processed for immunohistochemistry and tracing analysis. A set of 12 animals subjected to SCI at 3, 7, and 10 days (n=4 for each time point) was used for proteomic analyses.

Animals.

The study was performed with the approval and according to the guidelines of the Institutional Animal Care and Use Committee of the Slovak Academy of Sciences and with the European Communities Council Directive (2010/63/EU) regarding the use of animals in Research, Slovak Law for Animal Protection No. 377/2012 and 436/2012. In the present study, we tested a total of 40 rats, of which 34 survived.

Spinal Cord Injury.

The SCI was induced using the modified balloon-compression technique according to our previous study. Briefly, 2-French Fogarty catheter was inserted epidurally at Th8-9 level, and the balloon was inflated with 12.5 μl of saline for 5 min After compression of spinal cord tissue, the catheter was deflated and removed from the epidural space. In the sham group (n=4), the catheter was inserted at the same level of the spinal cord, but the balloon was not inflated and no lesion was performed. Manual bladder expression was required for 7-14 days after the injury until the bladder reflex was established. No antibiotic treatment was used.

Preparation of Alginate Scaffold with Affinity-bound Factors.

Fabrication of the scaffold with the affinity-bound dual growth factors (ALG+GFs) involved preparing bioconjugates of bFGF and EGF with alginate-sulfate and then mixing both bioconjugate solutions with the solution of a partially calcium-cross-linked alginate. The bioconjugates were prepared by mixing bFGF or EGF with alginate-sulfate solution (1%, w/v) and incubating for 1.5 h at 37° C., to allow equilibrium binding. The partially calcium-cross-linked alginate solution was prepared as previously described. Briefly, stock solutions of sodium alginate (VLVG, 30-50 kDa, >65% guluronic acid content, NovaMatrix FMC Biopolymers, Drammen, Norway) and D-gluconic acid/hemi calcium salt were prepared by dissolving the materials in DDW and stirring at room temperature. Each solution was filtered separately through a sterile 0.2 μm filter membrane into a sterile container in a laminar flow cabinet. Equal volumes from each stock solution (2.08% and 0.68% (w/v) for VLVG alginate and D-gluconic acid, respectively) were combined by extensive homogenization for several minutes to facilitate homogenous distribution of the calcium ions and cross linking of alginate chains Finally, the bFGF and EGF alginate-sulfate bioconjugates were mixed with the partially cross-linked alginate to yield an bFGF/EGF-containing, affinity-binding alginate scaffold (0.1% alginate-sulfate, 0.9% alginate, 0.3% D-gluconic acid, w/v) (ALG+GFs). For the control system lacking GFs, the scaffold was prepared with no affinity-bound factors (ALG).

Intraspinal Delivery of Alginate Scaffold.

Seven days after SCI, animals were anesthetized with 1.5-2% halothane and partial laminectomy at Th6-12 level was performed. Using a 50-μl Hamilton syringe (30G needle, Cole Parmer, Anjou, Quebec) connected to UltraMicroPump III with Micro4 Controller, 4-Channel (World Precious Instruments, Inc., Sarasota Fla.) and stereotactic device, 4 intraspinal injections/per animal were applied at the lesion site that showed discrete signs of haemorrhage and slight atrophy. In most cases the lesion cavity was apparent through the dorsal site of spinal cord. Bilateral delivery of i) saline, ii) ALG, or iii) ALG+GFs (2 injections of 2 μl/per injection/on left and 2 injections of 2 μl/per injection/on right side with delivery rate of 0.5 μl/min, loaded with 200 ng/ml of each GF) was performed. Based on our in vitro study results, an affinity-binding alginate scaffold loaded with 200 ng of bFGF/EGF confirmed long term release of GFs (Cizkova et al., J Tissue Eng Regen Med. 2015 August; 9 (8):918-29). Each delivery was positioned 1 mm from the spinal cord midline and injected at the depth of 1.8-2 mm from the pial surface of the spinal cord. The distance between injections was 1 mm, avoiding vessels. After injecting the dose of alginate scaffold, the needle was maintained in the tissue for an additional 30 seconds. No antibiotic treatment was administered to the subjects.

Anterograde Biotinylated Dextran Amine (BDA) Motor Corticospinal Tract (CST) Axon Tracing.

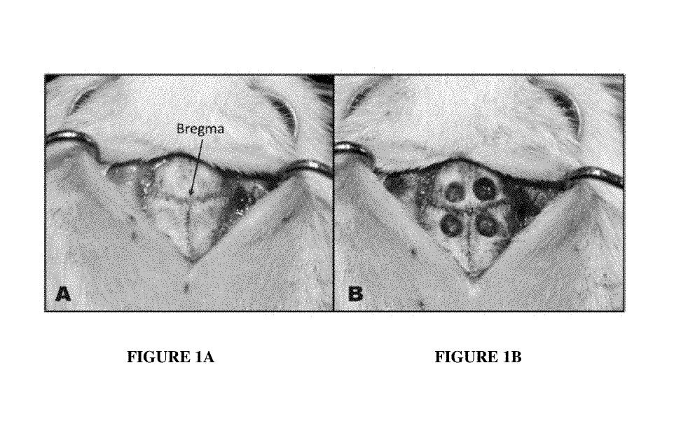

Sham rats (n=4) and SCI rats (ALG (n=3), and ALG+GFs (n=3)) at 3 weeks post-injury were anesthetized with 2% halothane and placed in a stereotaxic device. The halothane level was maintained at 2-3% throughout the surgery. An incision was made to expose the skull and to identify the bregma and lambda landmarks. Rats received injections of 10% solution of BDA (biotinylated dextran amine 10,000 MW; Molecular Probes, Eugene, Oreg.) in sterile 10 mM sodium phosphate buffer, pH 7.4, injected via glass micropipettes (inner tip diameter of 60-80 μm) using a controlled pressure device (PicoPump; World Precision Instruments). The injection site was positioned into right and left motor cortex performed at anatomical coordinates: 1.0 mm lateral to bregma, 1.5 mm anterior/posterior to the bregma and 1.5 mm deep to the cortical surface from the pial surface of the brain based on the Stereotaxic Coordinates (Paxinos and Franklin, 2001) (FIG. 1). A total of 8 injections with approximately 0.5 μl of BDA was injected at each of the four sites at a rate of 80 nl/min during 6-7 min/per injection. The micropipette remained in place for 3 minutes following each injection. After the delivery was completed, the skin overlying the skull was sutured and rats returned to their cages.

Behavioral Testing.

BBB scoring. Animals were behaviourally tested for 5 min using BBB open-field locomotor test after SCI at day 1, 3, 5, 7, and then at weekly intervals. Each rat was tested for 5 min by two blinded examiners. BBB test measures locomotor outcome (hindlimb activity, body position, trunk stability, tail position and walking paw placement) of rats by BBB rating scale ranges from 0—no observable hindlimbs movements to a maximum of 21—plantar stepping, coordination and trunk stability as demonstrated by healthy rats.

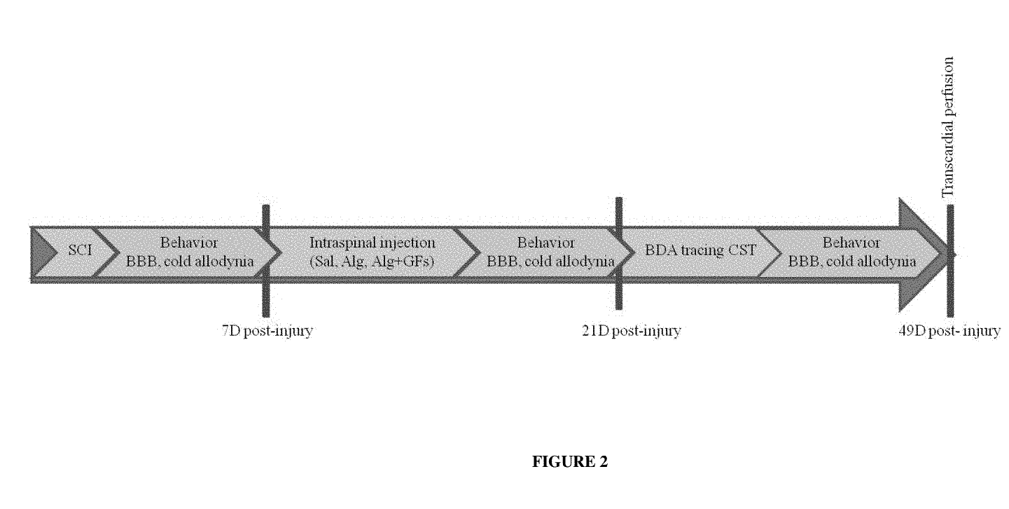

Cold Allodynia. Cold sensitivity of the hindpaws to acetone was quantified by foot withdrawal frequency. All animals were tested at day 3 post-injury and then in weekly intervals at day 16, 25, 32 and 49 after SCI. Before testing, the rats were left to acclimatize inside acrylic-plastic cages during the 10-15 min. A drop of acetone (50-100 μl) was applied to the left and right hindpaws using a plastic syringe, 5 times, with at least 5 min recovery between administrations. The number of brisk foot withdrawals or flinching were considered to be positive behaviours. Data are presented as mean response duration (in seconds). Statistical differences between groups were determined with an unpaired Student's t test. The sequence of surgical and behavioural procedures performed over time are described in FIG. 2.

### Tissue Processing and Immunohistochemistry.

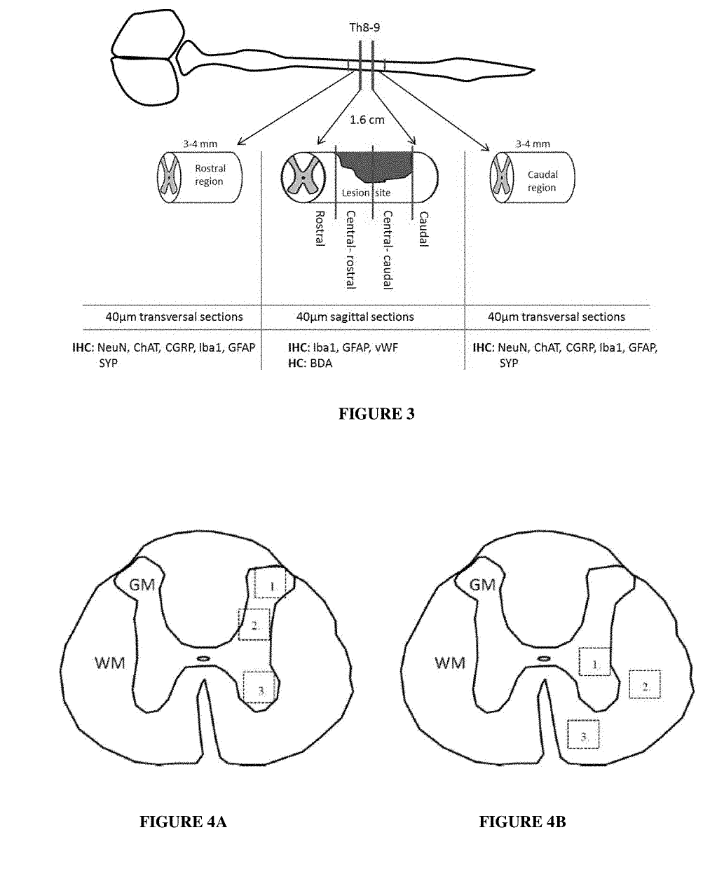

After a 49 day survival period, animals were deeply anesthetized by intraperitoneal thiopental injection (50 mg/kg) and perfused transcardially with 500 ml saline, followed by 500 ml of 4% paraformaldehyde (PFA) in 0.1 M phosphate-buffered. Spinal cords were removed, postfixed in 4% PFA at 4° C. overnight, embedded in gelatin-egg albumin protein matrix (10% ovalbumin, 0.75% gelatine) polymerized by glutaraldehyde (albumin from chicken egg white, grade II, Sigma-Aldrich) subsequently fixed in 4% PFA, and cryoprotected with 30% sucrose in 0.1 M PB at 4° C. Cryostat transversal and sagittal spinal cord sections (40 μm) were cut from rostral, central or caudal blocks (each 0.5 cm thick) (FIG. 3) and collected in 24-well plates with 0.1M PBS containing 0.1% sodium aside. For immunohistochemistry, free floating sections (40 μm) were immersed in PBS (0.1 M; pH 7.4) containing 10% normal goat or normal rabbit serum (NGS, NRS), 0.2% Triton X-100 for 2 h at room temperature to block non-specific protein activity. This was followed by overnight incubation at 4° C. with primary antibodies: mouse mouse anti-neuronal nuclei antigen (NeuN; 1:500, Merck-Millipore), goat anti-Choline acetyltransferase (ChAT; 1:5000, Merck-Millipore), rabbit anti-calcitonine gene related protein (CGRP; 1:100, Merck-Millipore), rabbit anti-ionized calcium-binding adapter molecule 1 (Iba1; 1:500, Wako), mouse anti-glial fibrillary acidic protein (GFAP; 1:1000, Merck-Millipore) rabbit anti-vWF (1:200, Chemicon) and mouse anti-synaptophysin (SYN; 1:500, Merck-Millipore) for 24 h. Afterwards, sections were washed in 0.1 M PBS and incubated with secondary fluorescent antibodies goat anti-mouse, goat anti-rabbit, rabbit anti-goat conjugated with Texas Red (Alexa Flour 594) and fluorescein isothiocyanate (FITC) (Alexa Flour 488) at room temperature for 2 hours. For general nuclear staining, 4-6-diaminidino-2-phenylindol (DAPI) (1:200) was added to the final secondary antibody solutions. Finally, sections were mounted and coverslipped with Vectashield mounting medium (Vector Laboratories).

Serial sagittal sections from each animal were stained for BDA to determine labelled CST axons through the lesion site. Sections were washed in TBS (50 mM Tris/HCl, 150 mM NaCl, pH=7.6) and subsequently incubated overnight at 4° C. with ABC kit (ABC Vectastain Ellite kit; Vector Laboratories) diluted in TBS. After rinsing 3 times for 10 minutes in TBS, sections were reacted with DAB (consisting of DAB substrate, DAB buffer and 0.6% NISO4) for 20 minutes. Sections were finally washed in TBS and coverslipped in Enthelan. Similarly, serial sagittal sections (n=5) were stained with Luxol Fast Blue to determine the length and cavity size area. A schematic concept of dissected spinal cord segments consisting of rostral (Th5-7), central (Th8-9), caudal (Th10-12) segments processed for immunohistochemical analyses is included in (FIG. 4).

Quantification Analysis.

Immunochemically stained sections were analyzed using Olympus BX-50 fluorescent microscope at 4×, 10× 20× and 40× magnifications, captured with digital camera HP Olympus and analyzed by Image J software according to the previous protocol. Quantification of NeuN, ChAT, Iba1, CGRP, GFAP and SYN positive cells was performed on five transverse sections from rostral and caudal segments of each spinal cord treatment and from sham tissue Similarly, for quantification of BDA tracing, GFAP, Iba1, vWF expression and cavity size in sagittal sections from lesion epicentre, five sections per each experimental animal were analyzed. Number of NeuN positive cells was evaluated through sample field of 200 μm×200 μm, bilaterally positioned at Laminae I-IV (DH=area 1), IV-V (deep dorsal horn=area 2) and VIII-IX (ventral horn=area 3). Analysis of ChAT labelling was performed by using the identical field positioned in Laminae VIII-IX, bilaterally. Number of Iba1+cells rostrally and caudally from injury epicenter was measured at the identical field in the Lamina VII gray matter (GM=area 1), lateral white matter (LWM=area 2) and ventral white matter (VWM=area 3) (FIGS. 4 A, B). Quantification of immunofluorescence intensity (CGRP, Iba1, SYN) rostrally/caudally from lesion site and GFAP, Iba1 also at the site of central lesion) was performed by using ImageJ software. Captured digital images were transformed into monochrome 8-bit images and determined the mean grey level number of black and white pixels within the tissue (value 0-255, when 0=black pixels, 255=white pixels). The final result yields the mean ratio of black and white pixels expressed by the histogram. Length of BDA were equally evaluated by Image J and expressed in mm Morphometric analyses of cavity size were performed on five 1.6 cm sagittal sections from the lesion site of each experimental and sham tissue. Modified Luxol Fast Blue labelling was performed to evaluate cavity area in spinal cord sections. Mean number of cavitation of experimental groups was expressed by mm relative to Sham spinal cord, which was without cavitation's and represent zero (no cavity).

Data and Statistical Analysis.

Obtained data from tissue analyses and behavioural testing were reported as mean±SEM. Mean values among different experimental groups were statistically compared by one-way ANOVA and Tukey's post hock tests using Graph pad PRISM software. Values of P<0.05 were considered statistically significant (*P value of <0.05, **P value of <0.01, ***P<0.001).

### Results

Locomotor Function Recovery.

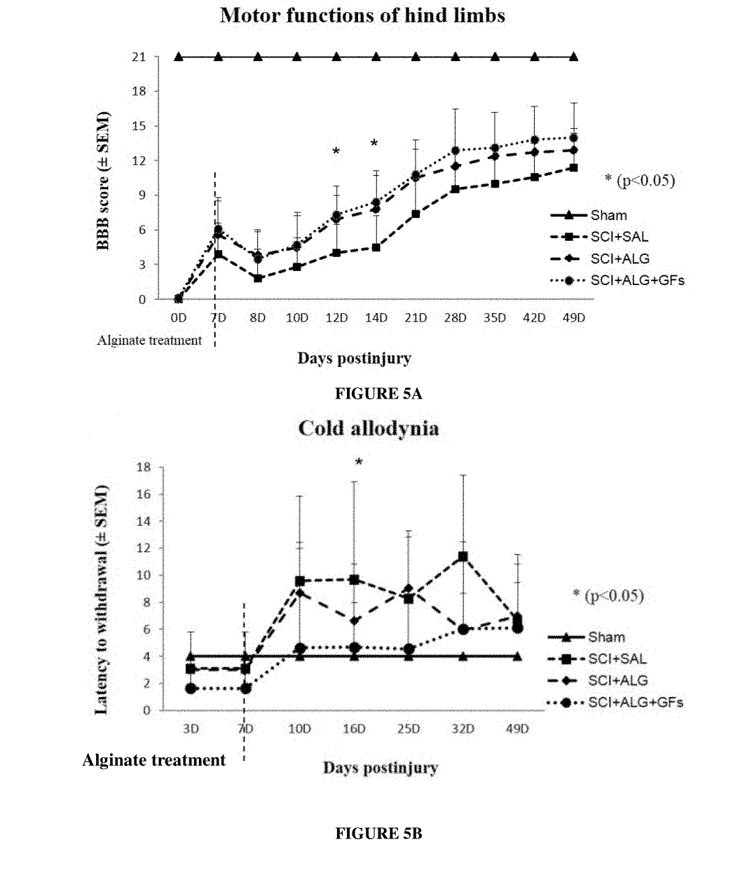

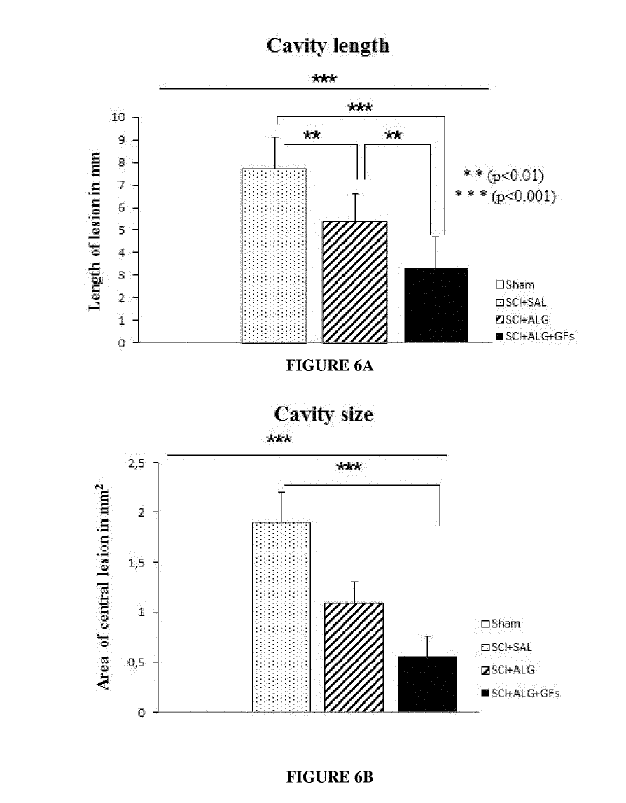

During the initial days post-injury, the compression caused hindlimb paralysis with slight movement in one or two joints in all experimental groups. On following days, the animals in the SCI+ALG and SCI+ALG+GFs biomaterial treatment groups showed a similar gradual recovery of hindlimb locomotion up to 21D after SCI; greater than the recovery in the SCI+SAL group, where only limited recovery of motor function was noted. The significant locomotor improvement (*p<0.05) between SCI+SAL and SCI+ALG/SCI+ALG+GFs was detected at 12 and 14 days post-injury with the final BBB scores 7.3±2.5/12D, 8.4±2.7/14D (SCI+ALG+GFs), 6.9±2.1/12D, 7.8±2.9/14D (SCI+ALG) and 4±2.4/12D, 4.5±2.714D (SCI+SAL) (FIG. 5A). The BBB score of hindlimb motor function gradually increased in all experimental groups with the survival (21, 28, 35, 42 and 49 days post-surgery), however the highest scores were observed in SCI+ALG+GFs (10.8±3, 12.9±3.6, 13.1±1.2, 13.8±3.1, 14.0±3), which closely correlated with SCI+ALG (10.5±2.5, 11.5±1.4, 12.4±0.9, 12.7±0.9, 12.9±1.9) but the increase was less prominent. The SCI+SAL group following initial gradual motor function improvement at 8 to 28 days, showed only limited recovery during further survival (7.4±3.6/21D, 9.5±3.6/28D, 10±3.1/35D, 10.6±2.9/42D, 11.4±3/49D) (FIG. 5A).

Cold Allodynia.

Increased sensitivity to normally non-painful cold stimulus is a characteristic feature of clinical neuropathic pain states. In our experiments, spinal cord injury result into adverse pain behavior—development of cold allodynia with positive responses to cold stimulus as paw withdrawal, licking, lifting and shaking of the hindpaw. Increased sensitivity to the acetone application developed mainly at 10D post-injury in the saline (9.6±6.2/SCI+SAL) and alginate (8.7±7.8/SCI+ALG) treated groups. Low increase in sensitivity to a normally innocuous stimulus was observed in SCI+ALG+GFs during entire survival (4.6±3.1/10D, 4.7±3.8/16D, 4.6±2.1/25D, 6±2.7/32D, 6.1±3.2/49D), compared to sham (4±1.8/10, 16, 25, 32, 49D). Frequent and more severely aversive responses to the acetone stimulus retained in SCI+ALG group at 10D, 16D, 25D (8.7±7.8/10D, 6.6±3.3/16D, 9±8.3/25D, 5.9±2.6/32D) and SCI+SAL group until 49D post injury (9.6±6.4/10D, 9.7±7.2/16D, 8.3±5/25D, 11.4±6/32D, 6.7±4.8/49D). Significant differences *p<0.05) were observed among SCI+SAL (9.7±7.2) and SCI+ALG+GFs (4.7±3.8) groups at 16D post-injury (FIG. 5B).

Cavity Size.

During the first week post-injury, a severe inflammatory response occurs at the central lesion. The secondary damage processes lead to cell death and development of cavitations at the epicenter and along the rostrocaudal axis of the spinal cord. In order to fill the cavity and create a permissive environment for regeneration, we administered the liquid form of alginate scaffold directly to the lesion cavity at 7D post injury.

Histological assessment of spinal cord sections stained with Luxol Fast Blue revealed cavity area reduction in SCI+ALG+GFs and SCI+ALG groups compared to SCI+SAL at 42 day post-implantation (FIG. 6). Quantitative stereological analyses of tissue fenestration in 1.6 cm segment revealed significant (**P<0.01, ***P<0.001) reduction of cavitation (analysing length and area of cavity) after application of ALG+GFs (cavity length/3.3±1.5 mm/area/0.56±0.2 mm²) and ALG (cavity length/5.4±1.2 mm/area/1.13±0.2 mm²) compared to the saline treatment (cavity length/7.7±1.4 mm/area/1.96±0.3 mm²) (FIG. 6).

Quantification of NeuN.

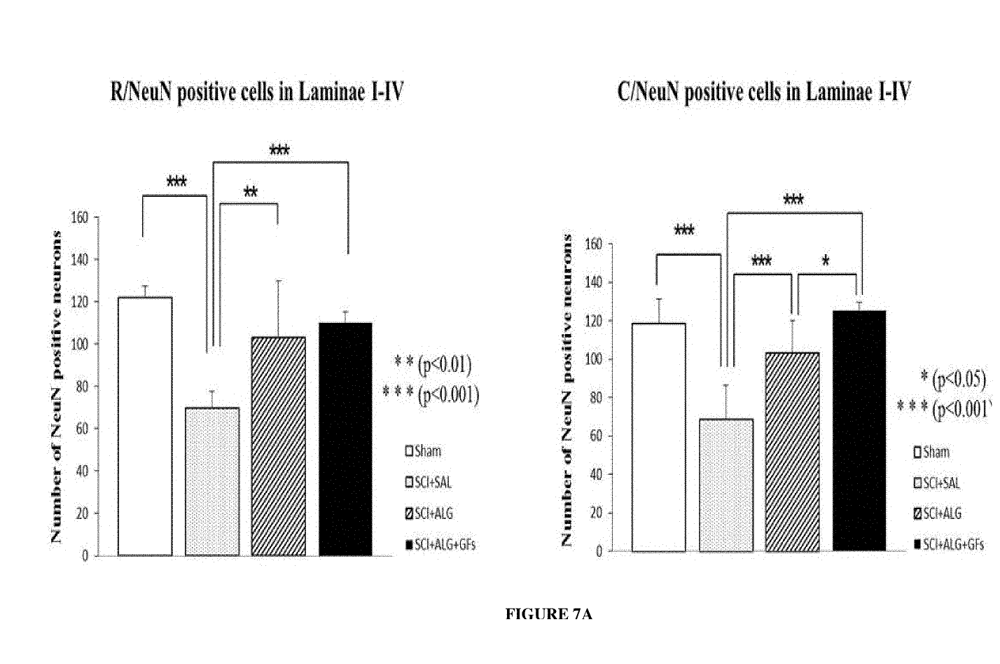

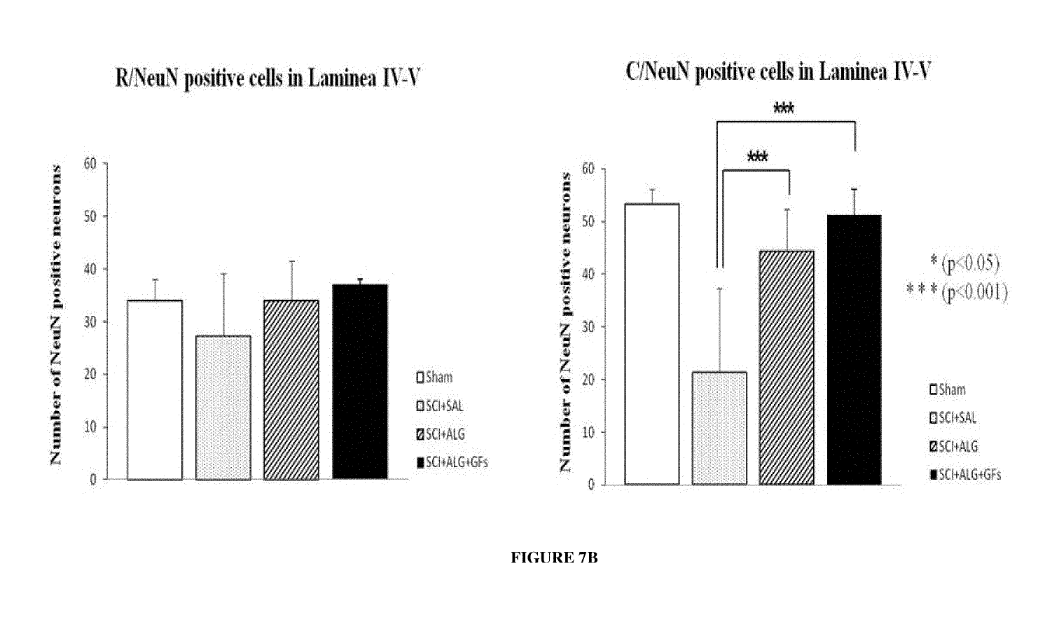

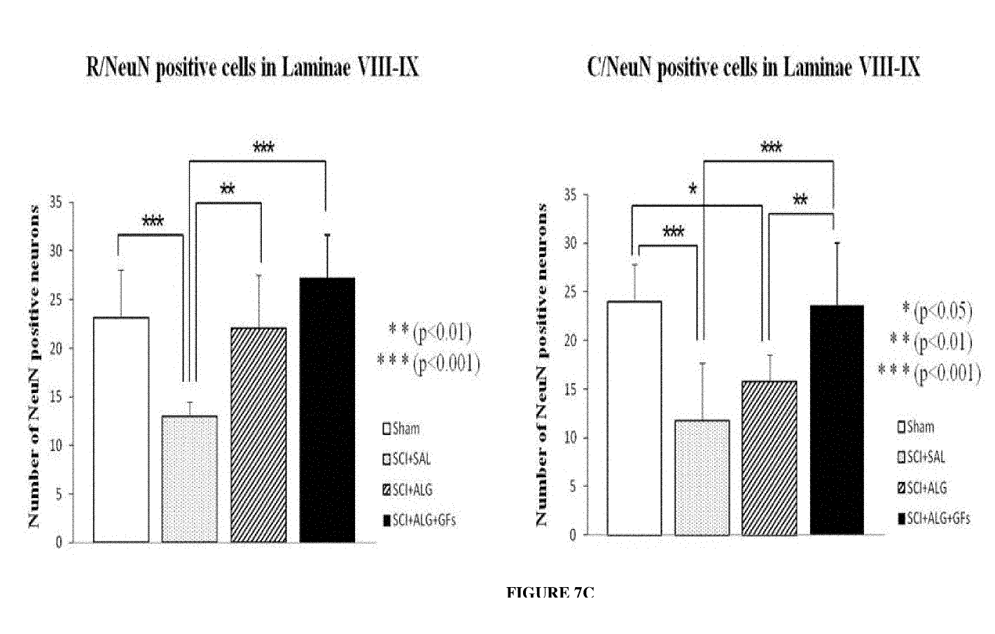

Quantification analyses of representative transverse sections were processed bilaterally in dorsal (Laminae I-IV), ventral horns (Laminae VIII-IX) and Laminae IV-V, rostrally and caudally from the lesion site (FIG. 7). The higher number of NeuN-positive cells was documented rostrally from the injury site in all studied groups SCI+ALG+GFs, SCI+ALG and SCI+SAL.

The most profound neuronal loss was observed in the SCI+SAL group (Rostral/Laminae I-IV 69.5±8.1; Laminae VIII-IX 13±1.5; Caudal/Laminae I-IV 68.8±17.7; Laminae VIII-IX 11.8±5.8). The delivery of ALG or ALG+GFs promoted the survival of neuronal cells, resulting in a significant increase in number of NeuN-positive cells SCI+ALG: Rostral/Laminae I-IV 103.1±26.6; Laminae VIII-IX 22±5.5; Caudal/Laminae I-IV 103.3±16.8; Laminae VIII-IX 15.8±2,7; SCI+ALG+GFs: Rostral/Laminae I-IV 110±5.3; Laminae VIII-IX 27.3±4.4; Caudal/Laminae I-IV 125.5±4.2; Laminae VIII-IX 23.6±6.4).

Moreover, the number of NeuN positive cells in the SCI+ALG+GFs group closely correlated with the NeuN numbers observed in Sham group (Rostral/Laminae I-IV 122±5.2; Laminae VIII-IX 23.1±5; Caudal/Laminae I-IV 120.7±12.7; Laminae VIII-IX 24±3.8) (FIG. 7). The differences in NeuN positive profiles show a statistical significance between individual experimental groups: Sham, SCI+SAL, SCI+ALG, SCI+ALG+GFs ***P<0.001, **P<0.01, *P<0.05.

ChAT Labeled Motoneurons.

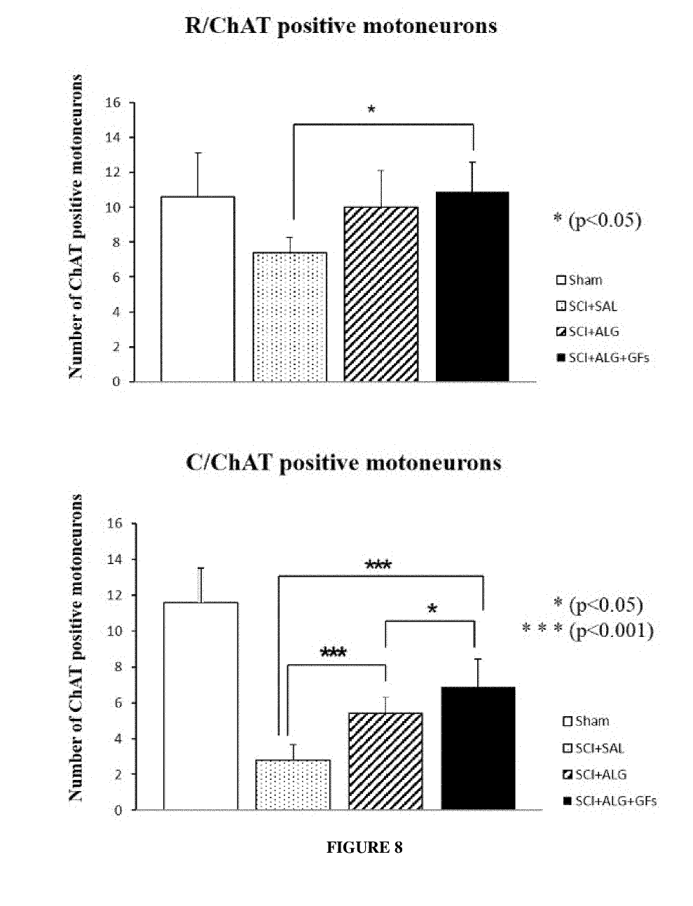

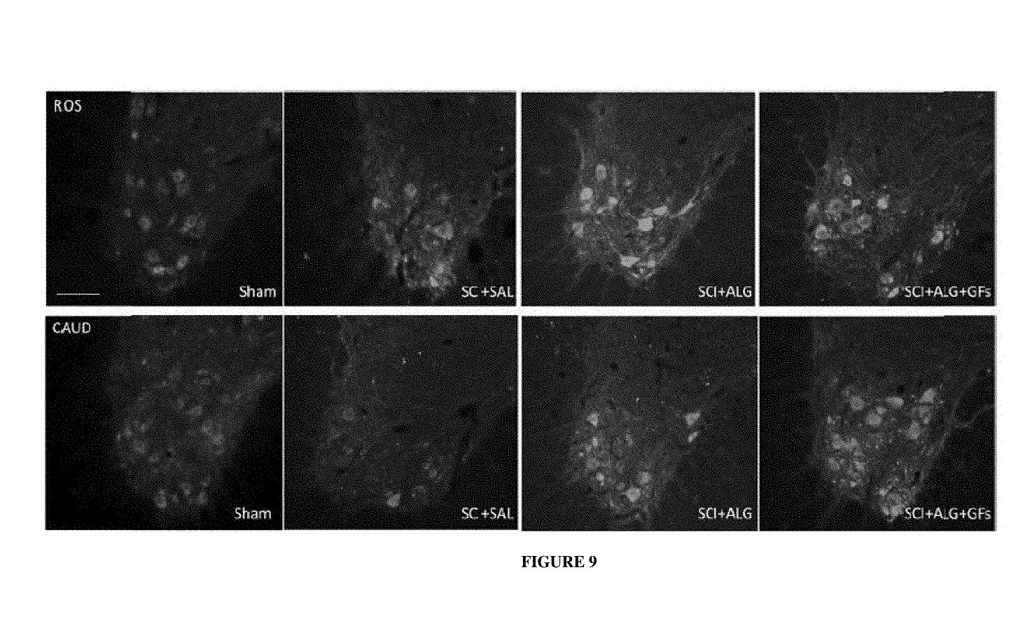

The average number of ChAT positive cells in the SCI+SAL, SCI+ALG and SCI+ALG+GFs groups was compared to confirm the hypothesis whether neuronal sparing has included motor neurons of ventral horns. Rostral to the lesion site, the number of spared ChAT+neurons within the ventral horns significantly increased (*P<0.05) following alginate biomaterial treatment (10±2.1/SCI+ALG; 10.9±1.7/SCI+ALG+GFs) when compared to the control saline group (7.4±0.9) (FIGS. 8 and 9). Significant differences in sparing of motor neurons (*P<0.05, ***P<0.001) were also recorded among experimental groups caudal to the injury site, although the average number of positive cells had declined (6.9±1.5/SCI+ALG+GFs, 5.4±0.9/SCI+ALG, 2.8±0.8/SCI+SAL, 11.9±1.9/Sham) compared to spinal rostral part (FIGS. 8 and 9). Our results demonstrate that alginate biomaterial implantation resulted not only in common NeuN positive neurons sparing, but also in the specific sparing of endogenous ChAT+motor neurons.

Synaptic vesicles alterations.

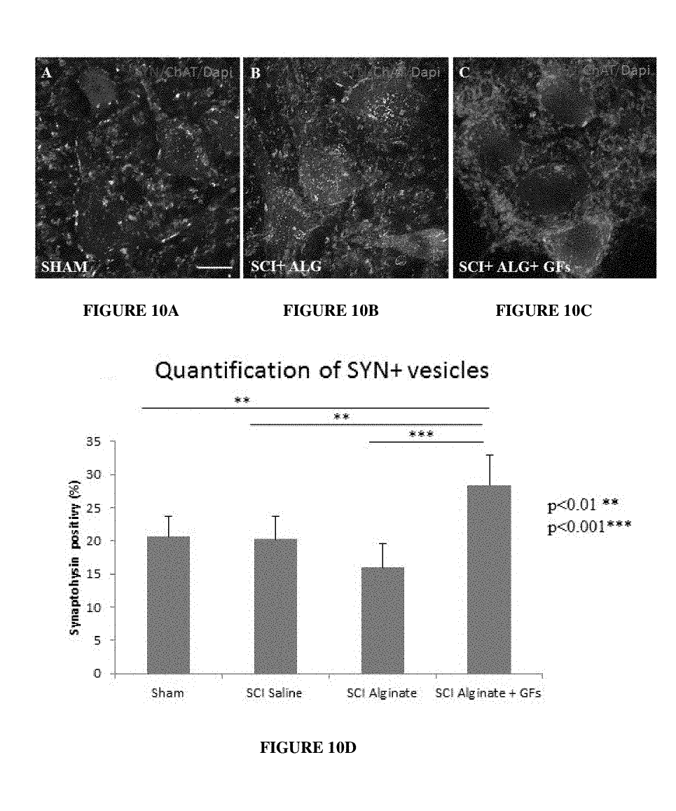

In the spinal cord of Sham and both SCI-SAL and SCI-ALG groups of treated rats, synaptophysin immunoreactivity (SYN+IR) appeared as numerous diffusely distributed fine dots along the surface of motor neurons and their proximal dendrites, and delineated their polygonal contours (FIGS. 10A, B). However, after ALG+GFs treatment, the density of SYN+vesicles around remaining CHAT+motor neurons of the anterior horns strikingly increased when compared to all experimental groups (FIGS. 10C, D). The immunoreactive profiles appeared as coarse granules of different size that were also distributed on motor neuron surface. Quantitative analysis of SYN+vesicle expressed as % of SYN+positive vesicles within identical fields of anterior horns in all experimental groups confirmed significant increase in ALG+GFs treated group, particularly caudally to the epicentre of injury (FIG. 10D). Interestingly, we did not see any differences in the density of SYN+positive vesicles within segments above the lesion site.

CGRP Positive Fibres.

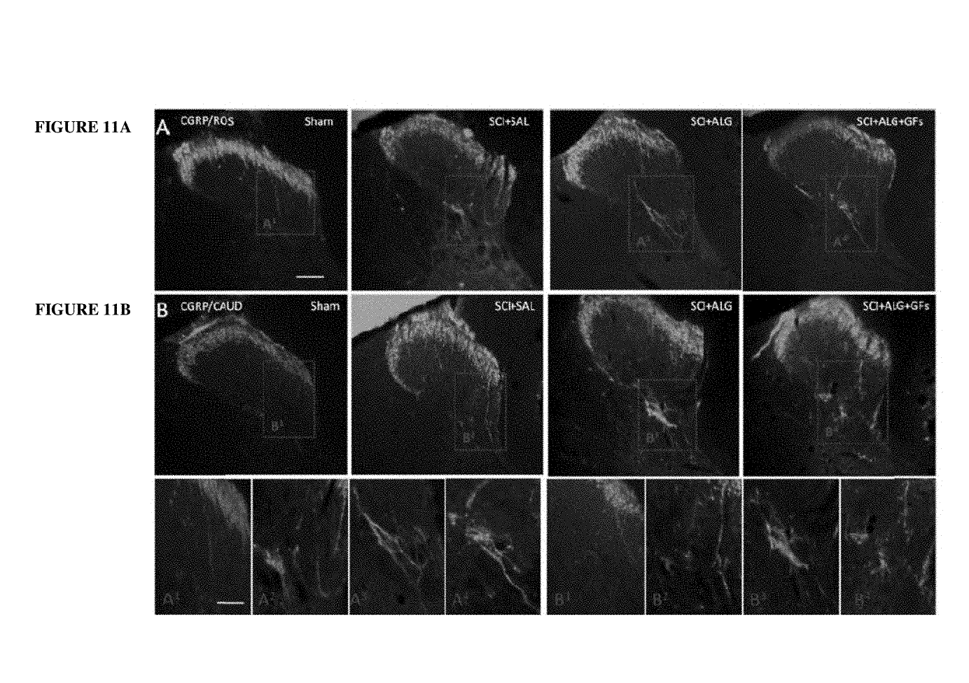

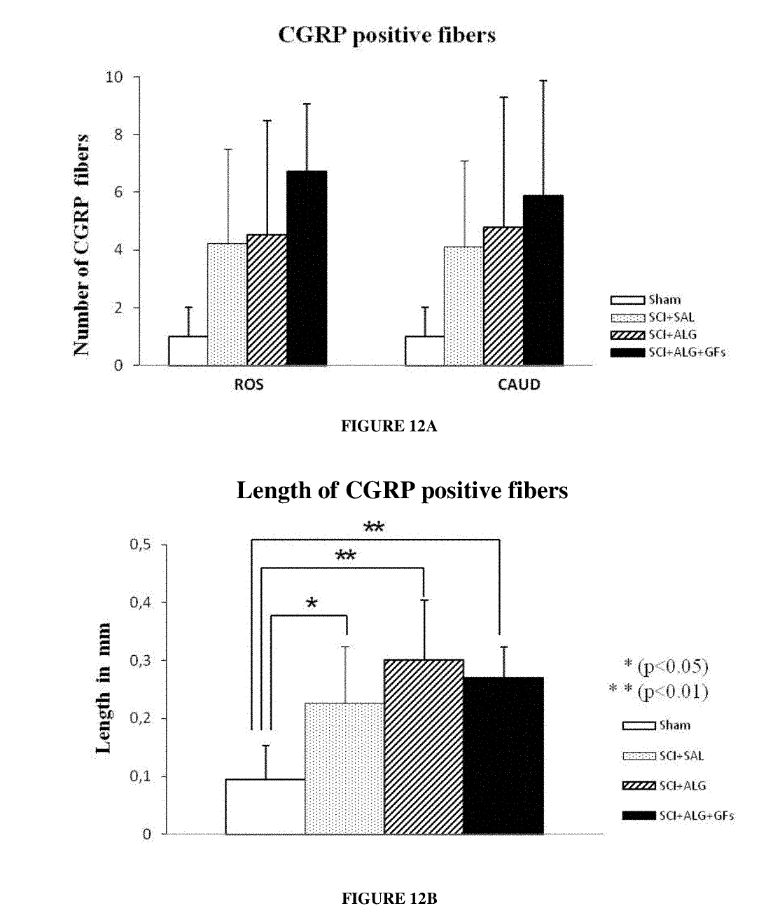

CGRP immunoreactivity was observed in all experimental groups (SCI+ALG+GFs, SCI+ALG, SCI+SAL) in fibres and punctuate terminals of superficial dorsal horn (Laminae I-III) and LT (LT-Lissauer's tract) area located along the lateral edge of the dorsal horn and medial grey mater (FIG. 11). Moreover, depending on the experimental group, few individual CGRP positive fibres extending from Lamina III toward Laminae V (0.226 ±0.099 mm/SCI+SAL) and VII (FIGS. 11 and 12) were detected. The longest CGRP+fibres with the average of length 0.301±0.103 mm were observed after administration of alginate biomaterial alone and alginate biomaterial with affinity-bound GFs to the injured spinal cord (0.301±0.103 mm/SCI+ALG; 0.27±0.053 mm/SCI+ALG+GFs) Sham spinal cord didn't contain CGRP positive fibres extended into the intermedia spinal cord layers; however CGRP terminals within superficial dorsal horn were frequently observed. The differences associated with length of fibres show statistical significance (**P<0.01, *P<0.05) only between Sham and other experimental groups (SCI+SAL, SCI+ALG, SCI+ALG+GFs) (FIG. 12).

The number of immune-labeled CGRP fibres varied among individual experimental groups and areas of spinal cord. The most numerous CGRP positive fibres, forming bundle-like structures were observed in rostral segments from the lesion site after the delivery of alginate biomaterial with the affinity-bound GFs (6.7±2.3/SCI+ALG+GFs, 4.5±4/SCI+ALG, 4.2±3.3/SCI+SAL, 1±1/Sham). The average number of positive fibres was slightly decreased caudally to the epicentre of injury (5.9±4/SCI+ALG+GFs, 4.8±4.5/SCI+ALG, 4.1±3/SCI+SAL, 1±1/Sham) (FIGS. 11 and 12). Among the individual experimental groups in both studied parameters we did not observe statistical differences (FIG. 12).

Axonal Sprouting via BDA Tracing.

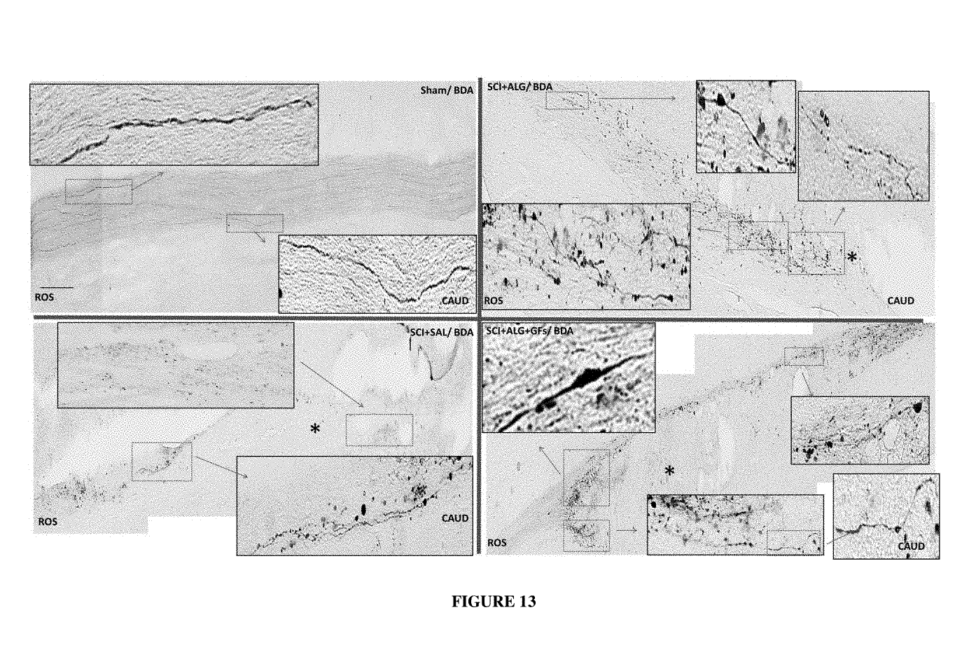

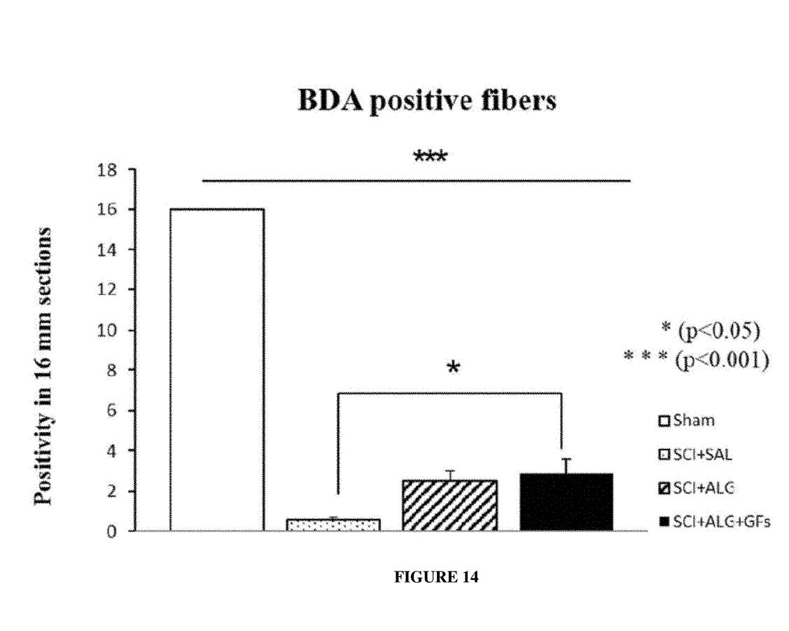

BDA delivery to the sensorimotor cortex served to label descending CST axons of spinal cord. In sham animals, BDA-labelled CST axons were detected along the entire length of sagittal sections (16 mm) of the spinal cord; more specifically in the ventral part of the dorsal column, where stripe of organized BDA positive axons occurred (16 mm±0) (FIGS. 13 and 14). After spinal cord injury, CST axons appeared disorganized, ended above the lesion site and many cut BDA axons formed terminal structures like buttons. Re-growth of CST fibres into denervated areas of spinal cord was monitored following alginate administration. Moreover, the alginate biomaterial alone and with affinity-bound EGF/bFGF promoted increased re-growth of few BDA positive fibres through the central lesion with occasional innervations below the lesion site (2.9 mm±0.7 from a total 16 mm length of section) compared to saline treatment (0.6 mm±0.1) (FIGS. 13 and 14) (*P<0.05).

Iba1 Immunohistochemistry.

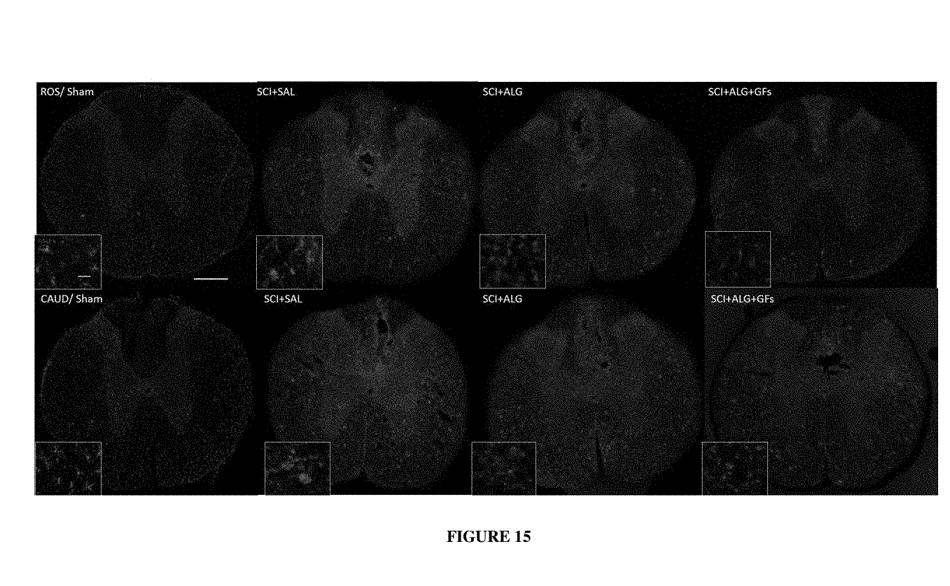

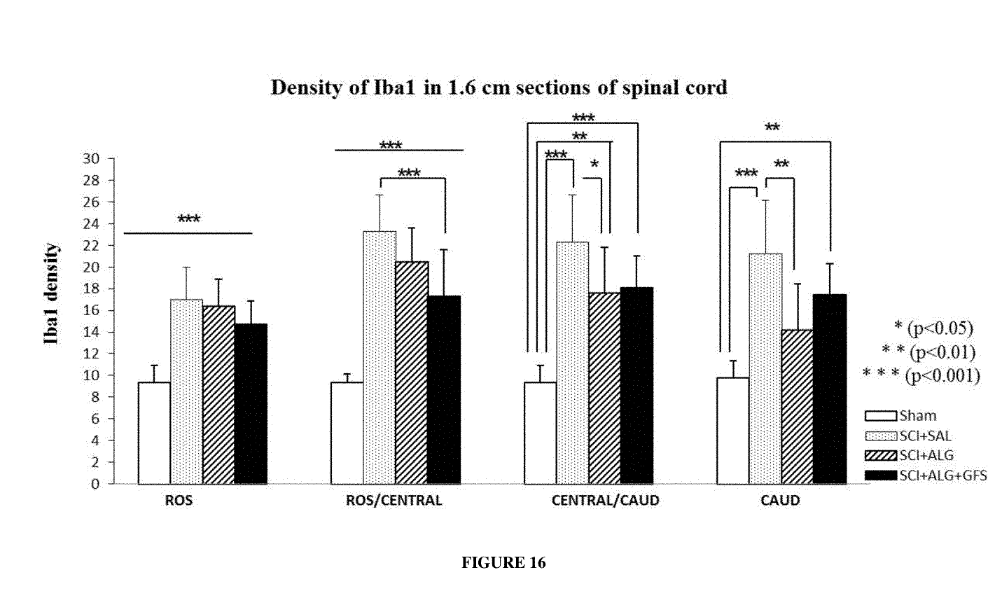

In order to monitor the immune response of host tissue, particularly the presence of microglia cells after alginate biomaterial treatment, Iba1 immunoreactivity in lesion site and also in the adjacent segments located 0.8 cm rostrally/caudally to the epicenter, were used Enhancement of the microglia responsiveness and subsequent density was observed mainly after injury with injection of saline (SCI+SAL: Ros/17±3, Ros-Centre/23.3±3.4, Centre-Caud/22.3±4.4, Caud/21.3±4.9), while the decreased tendency of Iba1 expression was seen after treatment with alginate biomaterial (SCI+ALG+GFs: Ros/14.8±2.1, Ros-Centre/17.3±4.3, Centre-Caud/18.1±3, Caud/17.4±2.9; SCI+ALG: Ros/16.4±2.5, Ros-Centre/20.5±3.2, Centre-Caud/17.6±4.2, Caud/14.2±4.3) (FIG. 15, FIG. 16). Positivity of Iba1 expression in Sham animals revealed basaline levels (Ros/9.4±1.5, Ros-Centre/9.3±0.8, Centre-Caud/9.3±1.6, Caud/9.8±1.6). Among experimental groups and individual parts of spinal cord significant differences were detected (***P<0.001, **P<0.01, *P<0.05, FIG. 16).

Glial Scar Modulation (GFAP Immunoreactivity).

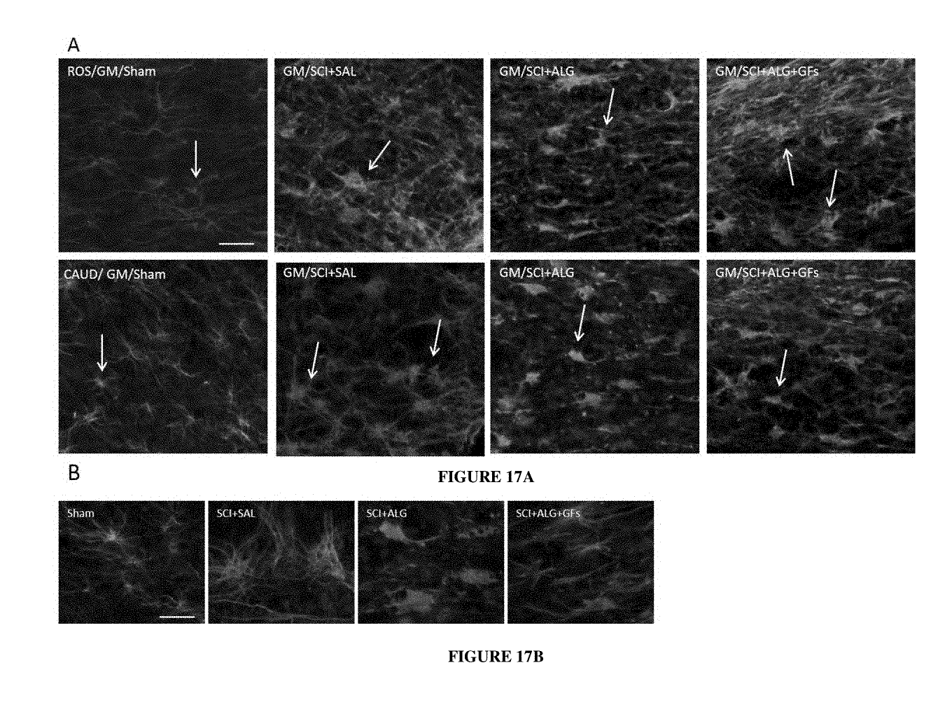

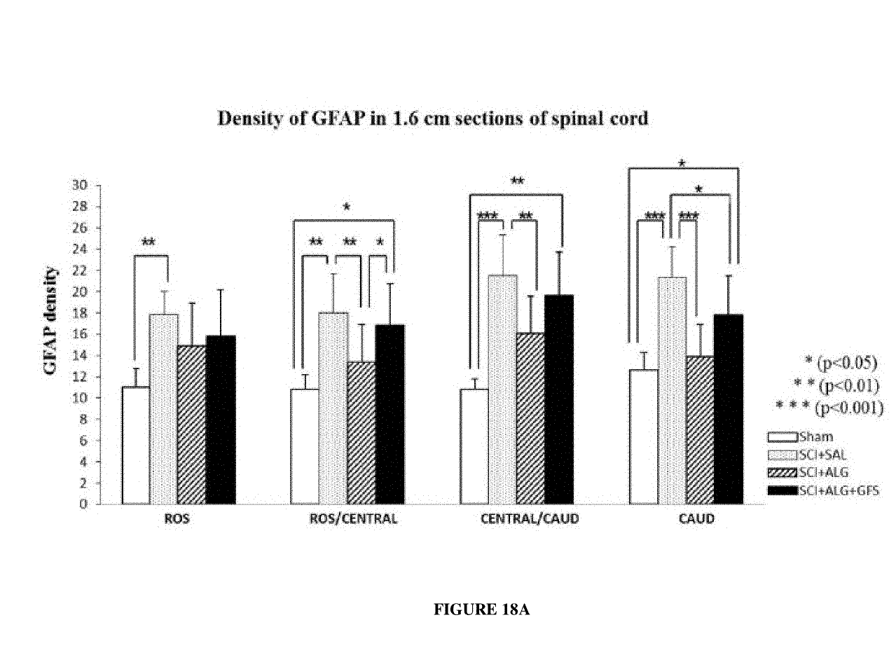

Baseline expression of GFAP-positive astrocytes with the characteristic round small soma and slender, long processes were seen in Sham spinal cord distributed throughout white and grey matter (Ros/11±1.8, Ros-Centre/10.81±1.41, Centre-Caud/10.81±1.41, Caud/12.6±1.6) (FIG. 17). The significant response of astrocytes that resulted in increased density and change of cellular morphology was observed following SCI and saline delivery (***P<0.001, **P<0.01, *P<0.05). Astrocytes assumed increased GFAP staining with subsequent cellular transformation into swollen hypertrophic appearance and short, thick processes indicating activated phenotype (FIG. 17) Similarly, alginate biomaterial treatment alone or with affinity-bound bFGF/EGF induced appearance of activated astrocytes, but with poorer ramification as seen after saline delivery (FIG. 17). The densitometry analysis revealed differences between the individual parts of 1.6 cm sections (Ros, Ros/Central, Central/Caudal, Caudal) of spinal cord.

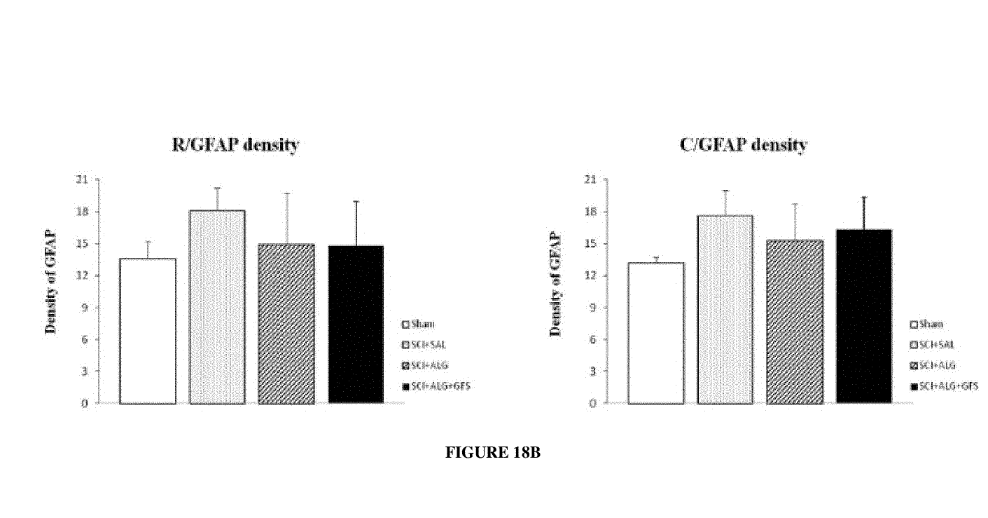

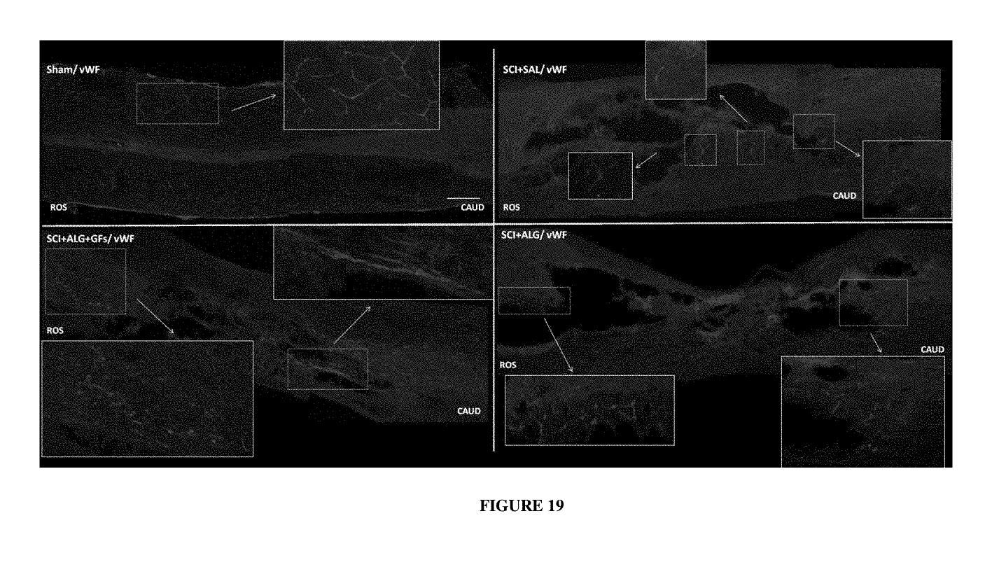

The highest positivity of GFAP was measured within Centro-Caudal site in all experimental groups (SCI+ALG+GFs: Ros/11±1.8, Ros-Centre/10.81±1.41, Centre-caud/10.81±1.41, Caud/12.6±1.6; SCI+ALG: Ros/14.9±4.1, Ros-Centre/13.4±3.6, Centre-Caud/16±3.5, Caud/13.8±3.1; SCI+SAL: Ros/17.9±2.2, Ros-Centre/18±3.7, Centre-Caud/21.5±3.9, Caud/21.4±1.6) (FIG. 18). The quantification of GFAP immunoreactivity on transverse sections from rostral and caudal segments of spinal cord also confirmed an increase in immunoreactivity caudally to the lesion site especially after saline delivery (Rostrally: 14.8±4.2/SCI+ALG+GFs, 14.9±4.9/SCI+ALG, 18.11±1.6/SCI+SAL, 13.6±1.6/Sham; Caudally: 16.3±3/SCI+ALG+GFs, 15.3±3.4/SCI+ALG, 17.6±2.4/SCI+SAL, 13.2±0.5/Sham) Differences in GFAP density between experimental groups in rostro-caudal segments were without observed significant differences (FIG. 18).

Angiogenesis.

For visualization of the vascular structures, the endothelial cell marker von Willebrand Factor (vWF) was used (FIG. 19). Numerous positive blood vessels were observed in the Sham group, mainly in the white matter compared to the injured spinal cord groups (SCI+SAL or SCI+ALG), where the density of blood vessels decreased in close vicinity of the lesion site (FIG. 19). Treatment with alginate biomaterial with the affinity-bound GFs resulted in an increase of vWF positive blood vessels in the white matter and in grey matter as well, at lesion site. Results obtained from immunohistochemical analyses suggest that GFs-enriched alginate biomaterial created a suitable environment for blood vessels survival or reconstruction, but without significant differences between treatment groups.

Currently, the field of SCI neurotherapeutics is still in its infancy and there are no effective and approved therapies for SCI in humans A contributing factor for such failed neuroregenerative processes has been attributed partly to the lack of development of an optimal regeneration-supportive microenvironment that can initiate a neurobridge connecting disconnected spinal cord segments.

The present study clearly demonstrates that the local delivery of injectable alginate biomaterial capable of increasing the bioavailability of key growth factors such as bFGF and EGF and their appropriate presentation improve the repair of SCI through multiple mechanisms, such as: i) reducing the central lesion cavity, ii) increasing the number of surviving neurons including ChAT+motor neurons and their synaptic connections, iii) enhancing outgrowth of CST axons, iv) preserving or stimulating formation of new blood vessels, and v) attenuating inflammation; which altogether enhance the functional recovery after SCI without sensory impairments.

Here we applied a well-characterized compression model of spinal cord injury leading to overall impairment of motor and sensory functions associated with loss of corresponding neuronal pools, overreaction of microglia/astrocytes and inability of axonal regrowth through the lesion site. Using a tissue micro-proteomic approach, we established that in the time period after lesion, the nature of proteins varied throughout the spinal rostro-caudal axis. Particularly, the proteins found in caudal and rostral segments at 7 and 10 days after SCI were different compared to 3 days post injury. Previous data clearly show that three days after lesion, the factors secreted in the lesion and rostral segments are anti-inflammatory and neurotrophic, while in the caudal region a cocktail of apoptotic and neurotoxic proteins predominate. The present study shows that on days 7 and 10 after SCI, in the caudal segments neurotrophic factors are overexpressed, as well as adhesion molecules and signaling proteins. In contrast, in rostral segments the proteins overexpressed are involved in metabolism at the level of the mitochondria or the cytoplasm, as well as in intracellular signaling. This clearly indicates that real differences exist between the rostral and caudal segments in terms of physiological and molecular processes, and that these differences are dynamic in time. Importantly, the results indicate that the caudal region possesses all the factors that can stimulate neurite outgrowth, but these seem to be insufficient in amount and are blocked by proteoglycans. Taking into account these ex vivo data, we attempted to connect the rostral and caudal segments through the lesion by constructing an alginate biomaterial bridge loaded with GFs.

Thus, all immunohistochemical and tracing analyses were performed along the rostro-caudal axis, to better understand and define differences in pathological or regenerative processes above and below the lesion site after biomaterial treatment.

Our strategy is in line with recent pre-clinical studies performed after incomplete/complete injuries, and attempting to reconnect links with the tissue below the injury site, either bypassing the central lesion or rebuilding tissue in a cyst mediated via the application of biomaterials. The novelty of our strategy is the combination of biomaterial used as a bridge together with sustained delivery of key growth factors for SCI repair. In vitro, this combination was found to be effective in promoting cell retention and expansion, while also enabling neural progenitor lineage differentiation in situ. In continuity with these findings, our in vivo results document significant spinal cord tissue sparing, resulting in neuronal sparing that may lead to enhanced plasticity and reorganization of preserved neuronal circuits. Furthermore, the sparing of ChAT positive motor neurons may correlate with the trend of motor function improvement observed during the whole survival period in SCI rats treated with the alginate scaffold with or without GFs, in comparison to animals treated with saline.

Physiological locomotion is governed by motoneurons that receive synaptic inputs from local interneurons, descending pathways and proprioceptive sensory neurons. The convergence of proper excitatory and inhibitory inputs on motoneurons mediated by synaptic connections is required for motor control, reflexes and tonic firing of the motoneurons. Disruption of the cellular components and/or synaptic connectivity in this spinal circuitry has been implicated in motoneuron spasticity and various motoneuron disorders. For this reason in this study we followed the response of ChAT motor neuron-related synaptic vesicles in segments above and below the central lesion using synaptophysin immunohistochemistry (SYN+IR). Synaptophysin is the most abundant integral membrane protein of synaptic vesicles and can be used as a marker protein of synaptic vesicles in the central and peripheral nervous systems. The present data document that SYN+IR around motor neurons in the anterior horns showed similar patterns in most experimental groups, except for the group receiving ALG+GFs. In these rats, we observed more intense SYN+IR in the caudal compared to the rostral segments. These results may be linked with our proteomic data, confirming the higher expression of neurotrophic and synaptogenetic factors in the caudal segment, thus producing a favourable environment for synaptic rebuilding reflected by increased SYN+IR after ALG+GF delivery. Although our proteomic findings respond to 10 days survival following SCI, the higher level of synaptogenetic factors may be further potentiated with a GF-enriched environment, as most likely seen in the present study with ALG+GF delivery. The mechanisms mediating increase in SYN+IR may reflect several processes such as: i) up-regulation of synaptic functions after SCI, which is more likely related to the release of excitatory amino acids, or ii) may indicate plastic changes associated with formation of new synapses. Thus, to further understand changes in motoneuron synaptic connectivity after SCI treatment, the transporter systems such as vesicular glutamate and glycine transporters (VGluT1/VGluT2, GlyT2) need to be further studied.

Furthermore, SCI-induced secondary pathological processes also cause interruption of the CST tract, leading to partial or complete impairment of descending motor pathways for skilled movements below the injury. The compression model used in the present experimental study carried out at thoracic levels caused interruption of axon fibres corresponding to both hindpaws with some degree of spontaneous regeneration and behavioural improvements. The behavioural outcome can be enhanced by promoting the axonal integrity and plasticity of the corticospinal tract and descending serotonergic pathways via GF delivery. In according with these finding, our data confirm significant re-growth of BDA positive fibres observed after intraspinal injection of GF-enriched alginate biomaterial at the central lesion. In contrast, delivery of alginate biomaterial alone did not induce the same effect as the GF-enriched biomaterial. The neuroprotective effect of biomaterial on axon regrowth has been described in many other in vitro and in vivo studies, where in vitro studies demonstrate that biomaterial promotes neural cell attachment and neurite outgrowth while in vivo studies show only partial regeneration after gel is implanted in the injured spinal cord. The explanation for differences in axonal outgrowth seen between in vitro and in vivo may be given by multiple factors associated with inhibitory, immune, endocrine processes that are typical for the complex in vivo environment. In addition, optimal regeneration of axons requires preserved vascular supply.

Our data indicate that an alginate scaffold may provide an appropriate substrate also for the survival and re-growth of blood vessels. Furthermore, growth factors affinity-bound to the alginate scaffold promote the survival, proliferation and differentiation of microvascular cells, which results in extensive collateral branching of damaged vessels and thickening of vessels within the lesion site.

Another important issue in damaged spinal cord pathology is the development of central sensitization, which often contributes to hyperalgesia and allodynia typically associated with inflammatory pain. In the present study therefore we addressed the response of sensory fibres expressing calcitonin gene-related peptide (CGRP) following treatment with alginate biomaterial. Our data show that significant increase in CGRP+fibres occurred in the dorsal horns and lateral grey matter after ALG+GF delivery. These most likely represent unmyelinated pelvic afferent fibres that convey thermal and nociceptive information. Plastic re-organization of spinal neural circuitry and morphological changes in the spinal reflex pathway (primary afferent fibres and spinal interneurons) may be responsible for serious post-injury complications that could lead to uncontrolled excitatory activity of glutamate-driven sympathetic preganglionic neurons, and similarly to loss of inhibitory GABAergic/glycinergic interneurons that could have an impact on increased bilateral hind limb sensitivity to cold. Although the administration of GF-enriched alginate biomaterial promoted extending of CGRP positive fibres, we did not observe adverse sensory response to cold, such as observed after saline or pure alginate delivery. Responses of the hind limbs were relatively stable in the SCI+ALG+GF treated group during the whole survival period, with intensity similar to that in the sham controls. From our results we can speculate that alginate biomaterial with affinity-bound growth factors enhanced changes in CGRP fibres, but without behavioural adverse sensory response.

Central sensitization of spinal neurons or neuronal hyper-responsiveness and alterations in behavioural pain thresholds may be also in close correlation with microglial activation, as pointed out in some recent studies. It is known that release of excitatory amino acids, interleukin-, and prostaglandin E275 by microglia actively participate in the generation of central sensitization after SCI. On the basis of this hypothesis we can conclude that significant microglia response after saline delivery could induce an increase in central sensitization of spinal neurons and promote the kind of adverse sensory response to cold detected in the present study. However, ALG-GF treatment causing attenuation of microglia may lead to normalization of sensory behaviour.

Reactive astrocytes together with microglia and meningeal fibroblasts are known to participate in scar formation, representing a mechanical and chemical barrier for nerve tissue regeneration. Significant differences in GFAP+IR between the experimental groups were observed spatially, mainly in the central and caudal segments. However, treatment with ALG and ALG+GFs did not attenuate the activation and proliferation of astrocytes after SCI, which may ultimately contribute to glia scarring at the central lesion site.

In the present experimental study, we tried to define the efficacy of usage of alginate itself as well alginate enriched with GFs for spinal cord repair. EGF and bFGF were selected due to their stable and high binding properties, as well as long-term sustained GF release from alginates, confirmed in vitro. Although these factors are important for their mitotic and partially differential properties for endogenous neural progenitors and their ability to accelerate neovascularisation, they may also contribute to astrogliosis and tissue scarring. Other neurotrophins such as brain-derived neurotrophic factor (BDNF), glial cell-derived neurotrophic factor (GDNF), and neurotrophin-3 (NT-3) with neuroprotective action, or VEGF, PDGF with angiogenic properties can be used for incorporation into one alginate device. In addition, simultaneous digestion of Chondroitin Sulfate Proteoglycans by chondroitinase ABC (ChABC) can be performed as well. From the surgical point of view, the alginate scaffold described herein was injected into irregular spinal cord cavities, where it adjusted itself into the cavity shape and in the presence of calcium ions could undergo gelation in situ. This type of non-invasive technique for vehicle administration is advantageous in particular when considering the fragility of the spinal cord site.

In sum, we describe here that an affinity-binding alginate scaffold with sustained presentation of bFGF/EGF has the potential to serve as a useful delivery vehicle in a certain model of SCI damage, with proven capability to promote neuronal sparing, modulate motoneuron synapses, enhance regrowth of BDA-positive CST fibres and the presence of vWF positive vessels at the injury site, as well as enhancing changes in CGRP fibres, but without behavioural adverse sensory response. Furthermore, combinatory-based local therapy using a biomaterial scaffold with neurotrophic factors, inhibitory molecules, enzymes that could digest deposits of extracellular matrix, e.g. chondroitin sulphate proteoglycans, as well as stem cells, may provide more advanced therapy for spinal cord rep air.

### EXAMPLE 2

### Advanced Therapy of Spinal Cord Injury

One of skilled in the art can develop alginate-based biomaterials that function as: i) natural “bridges” to stimulate nerve tissue re-building processes at the central lesion cavity, ii) carriers for sustained biosignal/growth factor delivery and cell therapy, iii) matrix for human mesenchymal stem cells (MSCs) which provide neurotrophic and anti-inflammatory protection.

Our strategy also exploits the principles underlying the anti-inflammatory effects of apoptotic cell clearance by macrophages based on the application of phosphatidylserine (PS)-presenting liposomes.

The functionalization and optimisation of alginate biomaterial may lead to the development of commercially viable products for SCI treatment. The ability of this scaffold to remodulate damaged nervous tissue can be preclinically evaluated in various animal models of SCI. Preclinical testing of alginate scaffolds can be carried out in combination with human MSCs (adipose tissue derived-MSCs or from umbilical cord), which can be prepared according to GMP (good manufacture practice).

Inventors of the instant application present a complete novel approach for the treatment of SCI. Association of GMP-hydrogel alginate and phosphatidylserine (PS) presenting liposomes at the lesion site to switch macrophages/microglia phenotype to M2 is completely novel for SCI treatment.

Inventors of the instant application present an advanced therapy of SCI, by developing smart biomaterials with multiple functionalities to improve regeneration in patients.

Development of smart biomaterials is a rapidly expanding field of regenerative medicine and tissue engineering, particularly for pathological events leading to severe degeneration with a need to rebuild the lost tissue by bridging the central lesion cavity and potentiating endogenous plasticity, providing appropriate biosignals and molecules for plasticity and axonal growth. In this context, we select the alginate hydrogel as a starting material as it can be functionalized to promote the bioactivity of growth factors by prolonging their half-life and modulating their delivery as well as cell retention/activity by immobilization of cell-adhesion peptides. Furthermore, the alginate biomaterial exhibits excellent biocompatibility and can be used in the form of an in situ gelling material thus being injectable at the lesion site. After proving its safety in Phase I/II clinical trials, the efficacy of the alginate hydrogels are currently being tested in a clinical trial in cardiac regeneration in post myocardial infarction. When functionalized with sulfate groups, the biomaterial releases multi-factorial nerve growth factors in vitro as well as in vivo at the site of injury in a very controllable and consistent manner When functionalized with adhesion peptides (RGD, YIGSR, etc.), the resulting hydrogel support attachment and growth of human stem cells. Moreover, there are constantly improving new methods for isolation and processing stem cells, using highly compatible, biodegradable materials seeded with cells or their combinatory constructs for the treatment of patients with SCI. It should be noted that adult hMSCs isolated from the bone marrow as well as from umbilical cord tissue (UCT) are an excellent candidate for the treatment of CNS injuries due to their trophic, immunomodulatory and anti-inflammatory effects. However, the survival and differentiation of stem cells into the right lineages significantly depends on the presence of growth and trophic factors, and bioactive molecules which are dramatically reduced after SCI. Therefore, biomaterials capable of releasing trophic factors may support survival of stem cells in vitro or in vivo. The success of regenerative medicine strategy for SCI therapy depends on several factors, including: i) the proper selection and processing of stem cells (adult autologous/allogeneic, easily accessible with multipotential characteristics), ii) the development of biomaterials able to increase the functional activity of GFs and hMSCs, etc.

Results

One of the major events at the beginning of the SCI is the development of acute inflammatory process characterized by fluid accumulation and the recruitment of immune cells and microglia. Microglial cells normally function as sentinel immune cells regulating tissue homeostasis in the adult central nervous system (CNS) and participate in pathological processes, orchestrating tissue remodeling. Their functions appear to be complex as they exhibit both neuroprotective and neurotoxic effects. When the CNS is injured or affected by diseases, the resident ramified microglia morphologically transform into activated microglia” or “reactive microglia” with retracted processes and enlarged cell bodies, accumulate at the affected site and release various bioactive substances. Some are cytotoxic or pro-inflammatory and others may aid survival and regeneration. In fact, macrophages are divided in two sub-populations i.e. the macrophages M1 and M2 type. The M1 macrophages type produces some pro-inflammatory mediators like TNF-α and nitric oxide (NO), whereas the M2 modulate the immune response through IL-10, IL-4 and IL-13 and is involved in tissues repair. Two sub-populations of monocytes also exist i.e. which can be discriminated at the transcriptomic level by the presence of NOS2, CIITA, IL12, IL6 over-expressed genes for M1 cells or CX3CR1, arginase 1, CD206, Ym1, Fizz1 genes for M2 cells. The M1 population is involved in inflammatory response whereas the M2 plays a role in the neuron-glia cross-talk and maintains the microglial cells in physiological conditions to interact with neurons through the Fractalkine linker.

Inventors have recently demonstrated that the uptake of phosphatidylserine (PS)-presenting liposomes by LPS-activated peritoneal macrophages up-regulated the anti-inflammatory phenotype, as reflected by the enhanced expression of CD206 (Mannose receptor), a greater secretion levels of the anti-inflammatory cytokines TGFβ and IL-10 and down regulation of the pro-inflammatory marker CD86. The anti-inflammatory macrophage phenotype after uptake of PS-presenting liposomes mimicked that of activated macrophages after the uptake of apoptotic cells. These factors have been previously shown to contribute to the immunosuppressive effects of apoptotic cells in vivo, as shown in IL-10-deficient mice. Importantly, the PS-presenting liposomes were capable of modulating the macrophage activation state at the infarct site, in myocardial infarct animal model. Based on such results in order to reverse the macrophages/microglia phenotype, inventors are testing the ability of PS-presenting liposomes to modulate the microglia/infiltrated macrophages at the lesion level and will confirm the uptake of PS-presenting liposomes by these cells via PS-receptor endocytosis since inhibition of the regular nonspecific phagocytosis by cytochalasin B will normally not affect their uptake.

For in vivo experiments, inventors have developed a functionalized scaffold based on alginate/alginate-sulfate with affinity-bound growth factors (Epidermal growth factor and fibroblast growth factor 2). This biomaterial is capable of controlling the presentation and sustained release of the growth factors (GFs). Neural progenitor cells isolated from 18 day-old rat embryo brains and seeded into the scaffold during preparation were found to proliferate and differentiate within the vehicle. A continuous release of both bFGF and EGF was noted for a period of 21 days. The scaffold has a potential to serve as a cell delivery vehicle, with proven capability to promote cell retention and expansion, while enabling NPC lineage differentiation in situ. In vivo tests with such biomaterial was performed by inventors and confirmed the data obtained in vitro. Injection at the lesion of such biomaterial enhanced the sparing of spinal cord tissue and increased the number of surviving neurons (choline acetyltransferase positive motoneurons) and sensory fibres. In addition, we document enhanced outgrowth of corticospinal tract axons and presence of blood vessels at the central lesion. Collectively, based on our data, we confirm that functional recovery was significantly improved in SCI groups receiving alginate scaffold with affinity-bound growth factors (ALG+GFs), compared to SCI animals without biomaterial treatment.

Biomaterials

One can develop alginate-based biomaterials that function as: (i) natural “bridges” to stimulate nerve tissue re-building processes at the central lesion cavity; (ii) carriers for sustained biosignal/growth factor delivery and cell therapy; and (iii) matrix for human stem cells derived from bone marrow or umbilical cord which provide neurotrophic and anti-inflammatory protection.

Our objectives exploit the principles underlying the anti-inflammatory effects of: 1) human mesenchymal stem cells (hMSCs), phosphatidylserine (PS)-presenting liposomes mimicking apoptotic cells and natural smart “bridges” to stimulate nerve tissue re-building processes at the central lesion cavity including hMSCs, which provide neurotrophic and anti-inflammatory protection.

The biomaterials will be able to compensate for the destroyed nerve tissue, attenuate inflammation, and stimulate neuroprotection, axonal regrowth and revascularization. The proposed neuron-targeted delivery system is expected to constitute a safer and specific delivery system as well as a more advanced concept in the context of SCI regenerative therapies.

Developing Injectable Alginate Biomaterial

One can develop an alginate biomaterial functionalized with sulfate groups, to enable sustained presentation/delivery of neurotrophins, to promote cell retention and activity of hMSCs, thus targeting neuroprotection, axonal growth and inflammation inhibition.

Mimicking the Anti-inflammatory Effects of Apoptotic Cell Clearance by Macrophages Based on the Application of PS-presenting Liposomes at the Lesion Site.

The exogenous application of PS-presenting liposomes as apoptotic-mimicking particles after acute SCI may modulate macrophage activity while providing a safe acellular, reproducible, and accessible approach. Our data document that macrophages share a similar machinery of apoptotic cell recognition; they both respond to the “eat me” signal presented as phosphatidylserine on apoptotic cell surface, and as a result they secrete similar patterns of cytokines. Based on these results, one can translate this strategy for the modulation of recruited or resident spinal microglia/macrophages after SCI by applying PS-presenting liposomes in vitro and in vivo. Thus, in the present project, we can study the behavior of activated spinal microglia/macrophages toward apoptotic cells.

Promoting Regeneration and Regrowth in Vitro.

Human mesenchymal stem cells (hMSCs) cultured in alginate hydrogel can be examined, and their viability, retention and differentiation properties can be studied.

Stimulating in Vivo Regenerative Processes and Plasticity in the Injured Spinal Cord.

One can evaluate whether the most promising smart hydrogel biomaterials, designed to sustain GF release would bridge the primary lesion (pseudocyst) and allow the transfer of regrowing fibers through the lesion. The most effective and potent biomaterial-based medical device, that are accessible and processible under GMP standards, can be tested in animal models of SCI.

### Methodology

Biomaterial Development

One can develop biomaterials seeded with hMSCs i) able to bridge the lesion site cavity. hMSCs possess immune-regulatory and neurotrophic factors that can modulate the resident and infiltrated macrophages. hMSCs derived from patients adipose-tissues (hASCs) or umbilical cord (UC) is the most promising material, since these cells exhibit pleiotropic immune regulatory activities.

Identifying and Characterizing the Immunodulators Sharing the Highest Efficiency by in Vitro Study.

MSC-cell based therapy versus cell-free treatment via secreted factors of MSCs can be tested on SCI-derived primary microglia culture. To achieve this goal, one can test an acellular approach via factors released from MSCs on and primary rat microglia derived from injured rat spinal cord (CD11b+cells separated in a magnetic field using MS columns). Particular focus can be given to find factors orientating the microglial cells from M1 to M2 activation state. On the other hand, stem cells are known to modulate inflammation. In fact, MSCs have become hot candidates for cellular proteins regulating cell survival, proliferation and differentiation to specific cell types as well as modulating inflammatory response. Thus, one can compare efficacy between cellular and cellular MSCs on SCI-derived primary microglia in order to quantify the cytokines, chemokines released and M1/M2 phenotyping.

Evaluating the Efficacy of Uptake of Phosphatidyl Serine (PS)-presenting Liposomes.

The efficacy of uptake of Phosphatidyl Serine (PS)-presenting liposomes by SCI primary microglia /macrophages culture can be evaluated by distinguishing the ratio of M1/M2 phenotype expression of CD86/CD206 (Mannose receptor), secretion levels of the anti-inflammatory cytokines TGFβ and IL-10 and the pro-inflammatory marker CD86 and CD4⁺CD25^{high} regulatory T cell recruitment.

Controlling the Activation State of Microglial Cells.

One can control the activation state of microglial cells (responsible for local inflammation at the lesion site) in order to attenuate acute inflammation. To achieve this goal, one can test factors released from smart biomaterials on organotypic slices, primary rat microglia and primary spinal cord culture. Particular focus can be given to find factors orientating the microglial cells from M1 to M2 activation state and invading inflammatory macrophages towards macrophages with neurotrophic properties. Quantification based on cytokine arrays can be done in order to quantify the released cytokines, chemokines and the base M1 or M2 genes i.e. (IL12, IL6, NOS2, Ciita) for M1 and (CX3CR1, arginase 1, CD206, Fizz1, Ym1) for M2.

Based on the in vitro test, one can identify and characterize the better strategy to reverse macrophages/microglia cells phenotype from M1 to M2.

Promotion of Regeneration and Regrowth in Vitro.

The main effect of the neuroprotection and neuroregeneration is mediated by specific neurotrophic molecules and cytokines that could be delivered via biomaterials or stem cells incorporated to the materials. The main impact can be on growth associated protein 43 (Gap43), known to be abundant in axonal growth cones, and other antibodies such as, SMI32, MAP2 and TUJ-1, NF-200. As in vivo, the gel can convey hMSCs, and internalization of these cells can be tested as well. Effects on neurite outgrowth can be quantitatively assessed in the presence of myelin/CSPG (inhibitory environment). From data obtained here, potent biomaterial-based medical device that are accessible and processible under GMP standards can be developed. This technology is expected to lead to convenient and portable smart materials (medical device) with applications in the regenerative medicine.

Validating the Stimulation of Regenerative Processes and Plasticity in Injured Spinal Cord in Vivo.

One can test whether a new generation of plasticity-inducing biomaterials carried out: by either selected candidate molecules (promoting anti-inflammatory, neurotrophic action), or stem cells embedded in biomaterials can mediate beneficial effect on sensory-motor functionality in rat spinal cord trauma model. Based on in vitro and ex vivo results, one can evaluate whether MSCs and PS-liposomes administration therapy can mediate beneficial effect on sensory-motor functionality in rat spinal cord trauma model. Here one can evaluate whether suppression of microglia-initiated inflammatory responses, and promoting CD4⁺CD25^{high} regulatory T cells recruitment and M2 macrophage phenotype above and below the lesion can be effective in reducing the central lesion, glial scar formation and finally improve functional recovery. One can involve a broad spectrum of cell biological, anatomical, behavioral and immunohistochemical analysis, studying the changes of neurite growth projection patterns of known tracts (corticospinal tract (CST) tracing with Biotinylated dextran-BD) to reveal synaptic changes after injury. Thus, one can evaluate whether suppression of microglia-initiated inflammatory responses, and promoting M2 macrophage phenotype above and particularly below the lesion can be effective in reducing the central lesion, glial scar formation and together with neurotrophic support can finally improve functional recovery. One can involve a broad spectrum of cell biological, anatomical, behavioural, imaging, immunohistochemical and electrophysiological analysis, studying the changes of neurite growth projection patterns of known to revealing synaptic changes after injury. All experiments using laboratory animals can be performed in accordance with the European Communities Council Directive regarding the use of animals in research and can be approved by the Ethics Committee of the particular Institutes.

It will be appreciated by those skilled in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

What is claimed is:

1. A method for repairing or regenerating damaged spinal cord tissue in a subject; treating a spinal cord injury in a subject; promoting the regrowth of one or more axons in a subject with a spinal cord injury; the method comprising the step of: administering to said subject a composition comprising a polysaccharide and a polypeptide operably linked to said polysaccharide, wherein said polysaccharide is a sulfated alginate or a sulfated hyaluronan, and wherein said polypeptide is a growth factor, thereby repairing or regenerating said damaged spinal cord tissue in said subject.

2. The method of claim 1, wherein said method comprises increasing the number of surviving neurons, sensory fibres, or a combination thereof in a subject with a spinal injury, the method comprising the step of: administering to said subject a composition comprising a polysaccharide and a polypeptide operably linked to said polysaccharide, wherein said polysaccharide is a sulfated alginate or sulfated hyaluronan, and wherein said polypeptide is a growth factor, thereby increasing the number of surviving neurons, sensory fibres, or a combination thereof in said subject.

3. The method of claim 1, wherein said growth factor is an epidermal growth factor (EGF), a basic fibroblast growth factor (bFGF), or a combination thereof.

4. The method of claim 1, wherein said growth factor is insulin-like growth factor-1 (IGF-1).

5. The method of claim 1, wherein said growth factor is linked to said sulfated alginate to form a bioconjugate nanoparticle.

6. The method of claim 1, further comprising the step of seeding one or more progenitor cells into said composition prior to administration.

7. The method of claim 1, wherein said composition comprising said linked polysaccharide and polypeptide comprises a supporting matrix.

8. The method of claim 7, wherein said matrix comprises a polymer selected from the group consisting of a polysaccharide, a protein, an extracellular matrix component, a synthetic polymer, and a mixture thereof.

9. The method of claim 8, wherein said polymer is alginate.

10. The method of claim 9, wherein said matrix is a hydrogel composition.

11. The method of claim 1, wherein said composition is a multi-compartment composition, wherein a neuroprotective polypeptide is present in the first compartment and an angiogenic polypeptide is present in the second compartment of said multi-compartment composition.

12. The method of claim 11, wherein Epidermal Growth Factor (EGF) is present in the first compartment and basic Fibroblast Growth factor (bFGF) is present in the second compartment of said multi-compartment composition.

13. The method of claim 1, wherein said step of administering comprises intraspinal administration.

14. A method for repairing or regenerating damaged spinal cord tissue in a subject; treating a spinal cord injury in a subject; or promoting the regrowth of one or more axons in a subject with a spinal cord injury, the method comprising the step of: administering to said subject a composition comprising an alginate, alginate sulfate, or hyaluronan sulfate, or a combination thereof, and lacking a bioactive polypeptide, thereby repairing or regenerating said damaged spinal cord tissue in said subject.

15. The method of claim 14, further comprising the step of seeding one or more progenitor cells into said composition prior to administration.

16. The method of claim 14, wherein said composition comprises a supporting matrix.

17. The method of claim 16, wherein said matrix comprises a polymer selected from the group consisting of a polysaccharide, a protein, an extracellular matrix component, a synthetic polymer, and a mixture thereof.

18. The method of claim 17, wherein said polymer is alginate.

19. The method of claim 16, wherein said matrix is a hydrogel composition.

20. The method of claim 14, wherein said step of administering comprises intraspinal administration.
